SEQUENCE LISTING (8 sequences)

SEQ ID NO: 1 - DNA, 4313 bp, Artificial Sequence
  Plasmid pZKUM
taatcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagtgctcaagctcgtggcagccaagaaaaccaacctgtgtgcttctctggatgttaccaccaccaaggagctcattgagcttgccgataaggtcggaccttatgtgtgcatgatcaaaacccatatcgacatcattgacgacttcacctacgccggcactgtgctccccctcaaggaacttgctcttaagcacggtttcttcctgttcgaggacagaaagttcgcagatattggcaacactgtcaagcaccagtaccggtgtcaccgaatcgccgagtggtccgatatcaccaacgcccacggtgtacccggaaccggaatcgattgctggcctgcgagctggtgcgtacgaggaaactgtctctgaacagaagaaggaggacgtctctgactacgagaactcccagtacaaggagttcctagtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacagtgctaatacgttgaactacttatacttatatgaggctcgaagaaagctgacttgtgtatgacttaat

SEQ ID NO: 2 - DNA, 13565 bp, Artificial Sequence
  Plasmid pZKL3-9DP9N
gtacggattgtgtatgtccctgtacctgcatcttgatggagagagctccggaaagcggatcaggagctgtccaattttaattttataacatggaaacgagtccttggagctagaagaccattttttcaactgccctatcgactatatttatctactccaaaaccgactgcttcccaagaatcttcagccaaggcttccaaagtaacccctcgcttcccgacacttaattgaaaccttagatgcagtcactgcgagtgaagtggactctaacatctccaacatagcgacgatattgcgagggtttgaatataactaagatgcatgatccattacatttgtagaaatatcataaacaacgaagcacatagacagaatgctgttggttgttacatctgaagccgaggtaccgatgtcattttcagctgtcactgcagagacaggggtatgtcacatttgaagatcatacaaccgacgtttatgaaaaccagagatatagagaatgtattgacggttgtggctatgtcataagtgcagtgaagtgcagtgattataggtatagtacacttactgtagctacaagtacatactgctacagtaatactcatgtatgcaaaccgtattctgtgtctacagaaggcgatacggaagagtcaatctcttatgtagagccatttctataatcgaaggggccttgtaatttccaaacgagtaattgagtaattgaagagcatcgtagacattacttatcatgtattgtgagagggaggagatgcagctgtagctactgcacatactgtactcgcccatgcagggataatgcatagcgagacttggcagtaggtgacagttgctagctgctacttgtagtcgggtgggtgatagcatggcgcgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcaggaatagacatcttcaataggagcattaatacctgtgggatcactgatgtaaacttctcccagagtatgtgaataaccagcgggccatccaacaaagaagtcgttccagtgagtgactcggtacatccgtctttcggggttgatggtaagtccgtcgtctccttgcttaaagaacagagcgtccacgtagtctgcaaaagccttgtttccaagtcgaggctgcccatagttgattagcgttggatcatatccaagattcttcaggttgatgcccatgaatagagcagtgacagctcctagagagtggccagttacgatcaatttgtagtcagtgttgtttccaaggaagtcgaccagacgatcctgtacgttcaccatagtctctctgtatgccttctgaaagccatcatgaacttggcagccaggacaattgatactggcagaagggtttgtggagtttatgtcagtagtgttaagaggagggatactggtcatgtagggttgttggatcgtttggatgtcagtaatagcgtctgcaatggagaaagtgcctcggaaaacaatatacttttcctttttggtgtgatcgtgggccaaaaatccagtaactgaagtcgagaagaaatttcctccaaactggtagtcaagagtcacatcgggaaaatgagcgcaagagtttccacaggtaaaatcgctctgcagggcaaatgggccaggggctctgacacaataggccacgttagatagccatccgtacttgagaacaaagtcgtatgtctcctgggtgataggagccgttaattaagttgcgacacatgtcttgatagtatcttgaattctctctcttgagcttttccataacaagttcttctgcctccaggaagtccatgggtggtttgatcatggttttggtgtagtggtagtgcagtggtggtattgtgactggggatgtagttgagaataagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgaccttttccttgggaaccaccaccgtcagcccttctgactcacgtattgtagccaccgacacaggcaacagtccgtggatagcagaatatgtcttgtcggtccatttctcaccaactttaggcgtcaagtgaatgttgcagaagaagtatgtgccttcattgagaatcggtgttgctgatttcaataaagtcttgagatcagtttggccagtcatgttgtggggggtaattggattgagttatcgcctacagtctgtacaggtatactcgctgcccactttatactttttgattccgctgcacttgaagcaatgtcgtttaccaaaagtgagaatgctccacagaacacaccccagggtatggttgagcaaaaaataaacactccgatacggggaatcgaaccccggtctccacggttctcaagaagtattcttgatgagagcgtatcgatggttaatgctgctgtgtgctgtgtgtgtgtgttgtttggcgctcattgttgcgttatgcagcgtacaccacaatattggaagcttattagcctttctattttttcgtttgcaaggcttaacaacattgctgtggagagggatggggatatggaggccgctggagggagtcggagaggcgttttggagcggcttggcctggcgcccagctcgcgaaacgcacctaggaccctttggcacgccgaaatgtgccacttttcagtctagtaacgccttacctacgtcattccatgcgtgcatgtttgcgccttttttcccttgcccttgatcgccacacagtacagtgcactgtacagtggaggttttgggggggtcttagatgggagctaaaagcggcctagcggtacactagtgggattgtatggagtggcatggagcctaggtggagcctgacaggacgcacgaccggctagcccgtgacagacgatgggtggctcctgttgtccaccgcgtacaaatgtttgggccaaagtcttgtcagccttgcttgcgaacctaattcccaattttgtcacttcgcacccccattgatcgagccctaacccctgcccatcaggcaatccaattaagctcgcattgtctgccttgtttagtttggctcctgcccgtttcggcgtccacttgcacaaacacaaacaagcattatatataaggctcgtctctccctcccaaccacactcacttttttgcccgtcttcccttgctaacacaaaagtcaagaacacaaacaaccaccccaacccccttacacacaagacatatctacagcaatggccatggccaaaagcaaacgacggtcggaggctgtggaagagcacgtgaccggctcggacgagggcttgaccgatacttcgggtcacgtgagccctgccgccaagaagcagaagaactcggagattcatttcaccacccaggctgcccagcagttggatcgggagcgcaaggaggagtatctggactcgctgatcgacaacaaggactatctcaagtaccgtcctcgaggctggaagctcaacaacccgcctaccgaccgacctgtgcgaatctacgccgatggagtgtttgatttgttccatctgggacacatgcgtcagctggagcagtccaagaaggccttccccaacgcagtgttgattgtgggcattcccagcgacaaggagacccacaagcggaagggattgaccgtgctgagtgacgtccagcggtacgagacggtgcgacactgcaagtgggtggacgaggtggtggaggatgctccctggtgtgtcaccatggactttctggaaaaacacaaaatcgactacgtggcccatgacgatctgccctacgcttccggcaacgacgatgatatctacaagcccatcaaggagaagggcatgtttctggccacccagcgaaccgagggcatttccacctcggacatcatcaccaagattatccgagactacgacaagtatttaatgcgaaactttgcccggggtgctaaccgaaaggatctcaacgtctcgtggctcaagaagaacgagctggacttcaagcgtcatgtggccgagttccgaaactcgttcaagcgaaagaaggtcggtaaggatctctacggcgagattcgcggtctgctgcagaatgtgctcatttggaacggcgacaactccggcacttccactccccagcgaaagacgctgcagaccaacgccaagaagatgtacatgaacgtgctcaagactctgcaggctcctgacgctgttgacgtggactcctcggagaacgtgtctgagaacgtcactgatgaggaggaggaagacgacgacgaggttgatgaggacgaagaagccgacgacgacgacgaagacgacgaagacgaggaagacgacgagtaggcggccgcattgatgattggaaacacacacatgggttatatctaggtgagagttagttggacagttatatattaaatcagctatgccaacggtaacttcattcatgtcaacgaggaaccagtgactgcaagtaatatagaatttgaccaccttgccattctcttgcactcctttactatatctcatttatttcttatatacaaatcacttcttcttcccagcatcgagctcggaaacctcatgagcaataacatcgtggatctcgtcaatagagggctttttggactccttgctgttggccaccttgtccttgctgtttaaacacgcagtaggatgtcctgcacgggtctttttgtggggtgtggagaaaggggtgcttggagatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgctatggaaccttgcggatatccatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaacatggccatcaaagtcggtattaacggattcgggcgaatcggacgaattgtgagtaccatagaaggtgatggaaacatgacccaacagaaacagatgacaagtgtcatcgacccaccagagcccaattgagctcatactaacagtcgacaacctgtcgaaccaattgatgactccccgacaatgtactaacacaggtcctgcccatggtgaaaaacgtggaccaagtggatctctcgcaggtcgacaccattgcctccggccgagatgtcaactacaaggtcaagtacacctccggcgttaagatgagccagggcgcctacgacgacaagggccgccacatttccgagcagcccttcacctgggccaactggcaccagcacatcaactggctcaacttcattctggtgattgcgctgcctctgtcgtcctttgctgccgctcccttcgtctccttcaactggaagaccgccgcgtttgctgtcggctattacatgtgcaccggtctcggtatcaccgccggctaccaccgaatgtgggcccatcgagcctacaaggccgctctgcccgttcgaatcatccttgctctgtttggaggaggagctgtcgagggctccatccgatggtgggcctcgtctcaccgagtccaccaccgatggaccgactccaacaaggacccttacgacgcccgaaagggattctggttctcccactttggctggatgctgcttgtgcccaaccccaagaacaagggccgaactgacatttctgacctcaacaacgactgggttgtccgactccagcacaagtactacgtttacgttctcgtcttcatggccattgttctgcccaccctcgtctgtggctttggctggggcgactggaagggaggtcttgtctacgccggtatcatgcgatacacctttgtgcagcaggtgactttctgtgtcaactcccttgcccactggattggagagcagcccttcgacgaccgacgaactccccgagaccacgctcttaccgccctggtcacctttggagagggctaccacaacttccaccacgagttcccctcggactaccgaaacgccctcatctggtaccagtacgaccccaccaagtggctcatctggaccctcaagcaggttggtctcgcctgggacctccagaccttctcccagaacgccatcgagcagggtctcgtgcagcagcgacagaagaagctggacaagtggcgaaacaacctcaactggggtatccccattgagcagctgcctgtcattgagtttgaggagttccaagagcaggccaagacccgagatctggttctcatttctggcattgtccacgacgtgtctgcctttgtcgagcaccaccctggtggaaaggccctcattatgagcgccgtcggcaaggacggtaccgctgtcttcaacggaggtgtctaccgacactccaacgctggccacaacctgcttgccaccatgcgagtttcggtcattcgaggcggcatggaggttgaggtgtggaagactgcccagaacgaaaagaaggaccagaacattgtctccgatgagagtggaaaccgaatccaccgagctggtctccaggccacccgggtcgagaaccccggtatgtctggcatggctgcttaggcggccgcatgagaagataaatatataaatacattgagatattaaatgcgctagattagagagcctcatactgctcggagagaagccaagacgagtactcaaaggggattacaccatccatatccacagacacaagctggggaaaggttctatatacactttccggaataccgtagtttccgatgttatcaatgggggcagccaggatttcaggcacttcggtgtctcggggtgaaatggcgttcttggcctccatcaagtcgtaccatgtcttcatttgcctgtcaaagtaaaacagaagcagatgaagaatgaacttgaagtgaaggaatttaaatagttggagcaagggagaaatgtagagtgtgaaagactcactatggtccgggcttatctcgaccaatagccaaagtctggagtttctgagagaaaaaggcaagatacgtatgtaacaaagcgacgcatggtacaataataccggaggcatgtatcatagagagttagtggttcgatgatggcactggtgcctggtatgactttatacggctgactacatatttgtcctcagacatacaattacagtcaagcacttacccttggacatctgtaggtaccccccggccaagacgatctcagcgtgtcgtatgtcggattggcgtagctccctcgctcgtcaattggctcccatctactttcttctgcttggctacacccagcatgtctgctatggctcgttttcgtgccttatctatcctcccagtattaccaactctaaatgacatgatgtgattgggtctacactttcatatcagagataaggagtagcacagttgcataaaaagcccaactctaatcagcttcttcctttcttgtaattagtacaaaggtgattagcgaaatctggaagcttagttggccctaaaaaaatcaaaaaaagcaaaaaacgaaaaacgaaaaaccacagttttgagaacagggaggtaacgaaggatcgtatatatatatatatatatatatacccacggatcccgagaccggcctttgattcttccctacaaccaaccattctcaccaccctaattcacaaccatggaggtcgtgaacgaaatcgtctccattggccaggaggttcttcccaaggtcgactatgctcagctctggtctgatgcctcgcactgcgaggtgctgtacctctccatcgccttcgtcatcctgaagttcacccttggtcctctcggacccaagggtcagtctcgaatgaagtttgtgttcaccaactacaacctgctcatgtccatctactcgctgggctccttcctctctatggcctacgccatgtacaccattggtgtcatgtccgacaactgcgagaaggctttcgacaacaatgtcttccgaatcaccactcagctgttctacctcagcaagttcctcgagtacattgactccttctatctgcccctcatgggcaagcctctgacctggttgcagttctttcaccatctcggagctcctatggacatgtggctgttctacaactaccgaaacgaagccgtttggatctttgtgctgctcaacggcttcattcactggatcatgtacggctactattggacccgactgatcaagctcaagttccctatgcccaagtccctgattacttctatgcagatcattcagttcaacgttggcttctacatcgtctggaagtaccggaacattccctgctaccgacaagatggaatgagaatgtttggctggtttttcaactacttctacgttggtactgtcctgtgtctgttcctcaacttctacgtgcagacctacatcgtccgaaagcacaagggagccaaaaagattcagtgagcggccgcaagtgtggatggggaagtgagtgcccggttctgtgtgcacaattggcaatccaagatggatggattcaacacagggatatagcgagctacgtggtggtgcgaggatatagcaacggatatttatgtttgacacttgagaatgtacgatacaagcactgtccaagtacaatactaaacatactgtacatactcatactcgtacccggcaacggtttcacttgagtgcagtggctagtgctcttactcgtacagtgtgcaatactgcgtatcatagtctttgatgtatatcgtattcattcatgttagttgc

SEQ ID NO: 3 - DNA, 777 bp, Euglena gracilis
  CDS (1)..(777): mutant delta-9 elongase “EgD9eS-L35G”
atggaggtcgtgaacgaaatcgtctccattggccaggaggttcttcccaaggtcgactatgctcagctctggtctgatgcctcgcactgcgaggtgctgtacgggtccatcgccttcgtcatcctgaagttcacccttggtcctctcggacccaagggtcagtctcgaatgaagtttgtgttcaccaactacaacctgctcatgtccatctactcgctgggctccttcctctctatggcctacgccatgtacaccattggtgtcatgtccgacaactgcgagaaggctttcgacaacaatgtcttccgaatcaccactcagctgttctacctcagcaagttcctcgagtacattgactccttctatctgcccctcatgggcaagcctctgacctggttgcagttctttcaccatctcggagctcctatggacatgtggctgttctacaactaccgaaacgaagccgtttggatctttgtgctgctcaacggcttcattcactggatcatgtacggctactattggacccgactgatcaagctcaagttccctatgcccaagtccctgattacttctatgcagatcattcagttcaacgttggcttctacatcgtctggaagtaccggaacattccctgctaccgacaagatggaatgagaatgtttggctggtttttcaactacttctacgttggtactgtcctgtgtctgttcctcaacttctacgtgcagacctacatcgtccgaaagcacaagggagccaaaaagattcagtga

SEQ ID NO: 4 - protein, 258 aa, Euglena gracilis
MEVVNEIVSIGQEVLPKVDYAQLWSDASHCEVLYGSIAFVILKFTLGPLGPKGQSRMKFVFTNYNLLMSIYSLGSFLSMAYAMYTIGVMSDNCEKAFDNNVFRITTQLFYLSKFLEYIDSFYLPLMGKPLTWLQFFHHLGAPMDMWLFYNYRNEAVWIFVLLNGFIHWIMYGYYWTRLIKLKFPMPKSLITSMQIIQFNVGFYIVWKYRNIPCYRQDGMRMFGWFFNYFYVGTVLCLFLNFYVQTYIVRKHKGAKKIQ

SEQ ID NO: 5 - DNA, 1449 bp, Yarrowia lipolytica
  CDS (1)..(1449): delta-9 desaturase; GenBank Accession No. XM_501496
atggtgaaaaacgtggaccaagtggatctctcgcaggtcgacaccattgcctccggccgagatgtcaactacaaggtcaagtacacctccggcgttaagatgagccagggcgcctacgacgacaagggccgccacatttccgagcagcccttcacctgggccaactggcaccagcacatcaactggctcaacttcattctggtgattgcgctgcctctgtcgtcctttgctgccgctcccttcgtctccttcaactggaagaccgccgcgtttgctgtcggctattacatgtgcaccggtctcggtatcaccgccggctaccaccgaatgtgggcccatcgagcctacaaggccgctctgcccgttcgaatcatccttgctctgtttggaggaggagctgtcgagggctccatccgatggtgggcctcgtctcaccgagtccaccaccgatggaccgactccaacaaggacccttacgacgcccgaaagggattctggttctcccactttggctggatgctgcttgtgcccaaccccaagaacaagggccgaactgacatttctgacctcaacaacgactgggttgtccgactccagcacaagtactacgtttacgttctcgtcttcatggccattgttctgcccaccctcgtctgtggctttggctggggcgactggaagggaggtcttgtctacgccggtatcatgcgatacacctttgtgcagcaggtgactttctgtgtcaactcccttgcccactggattggagagcagcccttcgacgaccgacgaactccccgagaccacgctcttaccgccctggtcacctttggagagggctaccacaacttccaccacgagttcccctcggactaccgaaacgccctcatctggtaccagtacgaccccaccaagtggctcatctggaccctcaagcaggttggtctcgcctgggacctccagaccttctcccagaacgccatcgagcagggtctcgtgcagcagcgacagaagaagctggacaagtggcgaaacaacctcaactggggtatccccattgagcagctgcctgtcattgagtttgaggagttccaagagcaggccaagacccgagatctggttctcatttctggcattgtccacgacgtgtctgcctttgtcgagcaccaccctggtggaaaggccctcattatgagcgccgtcggcaaggacggtaccgctgtcttcaacggaggtgtctaccgacactccaacgctggccacaacctgcttgccaccatgcgagtttcggtcattcgaggcggcatggaggttgaggtgtggaagactgcccagaacgaaaagaaggaccagaacattgtctccgatgagagtggaaaccgaatccaccgagctggtctccaggccacccgggtcgagaaccccggtatgtctggcatggctgcttag

SEQ ID NO: 6 - protein, 482 aa, Yarrowia lipolytica
MVKNVDQVDLSQVDTIASGRDVNYKVKYTSGVKMSQGAYDDKGRHISEQPFTWANWHQHINWLNFILVIALPLSSFAAAPFVSFNWKTAAFAVGYYMCTGLGITAGYHRMWAHRAYKAALPVRIILALFGGGAVEGSIRWWASSHRVHHRWTDSNKDPYDARKGFWFSHFGWMLLVPNPKNKGRTDISDLNNDWVVRLQHKYYVYVLVFMAIVLPTLVCGFGWGDWKGGLVYAGIMRYTFVQQVTFCVNSLAHWIGEQPFDDRRTPRDHALTALVTFGEGYHNFHHEFPSDYRNALIWYQYDPTKWLIWTLKQVGLAWDLQTFSQNAIEQGLVQQRQKKLDKWRNNLNWGIPIEQLPVIEFEEFQEQAKTRDLVLISGIVHDVSAFVEHHPGGKALIMSAVGKDGTAVFNGGVYRHSNAGHNLLATMRVSVIRGGMEVEVWKTAQNEKKDQNIVSDESGNRIHRAGLQATRVENPGMSGMAA

SEQ ID NO: 7 - DNA, 1101 bp, Yarrowia lipolytica
  CDS (1)..(1101): choline-phosphate cytidylyl-transferase; GenBank Accession No. XM_502978
atggccaaaagcaaacgacggtcggaggctgtggaagagcacgtgaccggctcggacgagggcttgaccgatacttcgggtcacgtgagccctgccgccaagaagcagaagaactcggagattcatttcaccacccaggctgcccagcagttggatcgggagcgcaaggaggagtatctggactcgctgatcgacaacaaggactatctcaagtaccgtcctcgaggctggaagctcaacaacccgcctaccgaccgacctgtgcgaatctacgccgatggagtgtttgatttgttccatctgggacacatgcgtcagctggagcagtccaagaaggccttccccaacgcagtgttgattgtgggcattcccagcgacaaggagacccacaagcggaagggattgaccgtgctgagtgacgtccagcggtacgagacggtgcgacactgcaagtgggtggacgaggtggtggaggatgctccctggtgtgtcaccatggactttctggaaaaacacaaaatcgactacgtggcccatgacgatctgccctacgcttccggcaacgacgatgatatctacaagcccatcaaggagaagggcatgtttctggccacccagcgaaccgagggcatttccacctcggacatcatcaccaagattatccgagactacgacaagtatttaatgcgaaactttgcccggggtgctaaccgaaaggatctcaacgtctcgtggctcaagaagaacgagctggacttcaagcgtcatgtggccgagttccgaaactcgttcaagcgaaagaaggtcggtaaggatctctacggcgagattcgcggtctgctgcagaatgtgctcatttggaacggcgacaactccggcacttccactccccagcgaaagacgctgcagaccaacgccaagaagatgtacatgaacgtgctcaagactctgcaggctcctgacgctgttgacgtggactcctcggagaacgtgtctgagaacgtcactgatgaggaggaggaagacgacgacgaggttgatgaggacgaagaagccgacgacgacgacgaagacgacgaagacgaggaagacgacgagtag

SEQ ID NO: 8 - protein, 366 aa, Yarrowia lipolytica
MAKSKRRSEAVEEHVTGSDEGLTDTSGHVSPAAKKQKNSEIHFTTQAAQQLDRERKEEYLDSLIDNKDYLKYRPRGWKLNNPPTDRPVRIYADGVFDLFHLGHMRQLEQSKKAFPNAVLIVGIPSDKETHKRKGLTVLSDVQRYETVRHCKWVDEVVEDAPWCVTMDFLEKHKIDYVAHDDLPYASGNDDDIYKPIKEKGMFLATQRTEGISTSDIITKIIRDYDKYLMRNFARGANRKDLNVSWLKKNELDFKRHVAEFRNSFKRKKVGKDLYGEIRGLLQNVLIWNGDNSGTSTPQRKTLQTNAKKMYMNVLKTLQAPDAVDVDSSENVSENVTDEEEEDDDEVDEDEEADDDDEDDEDEEDDE

(19) United States
(12) Patent Application Publication
(10) Pub. No.: US 2013/0046020 A1
(43) Pub. Date: Feb. 21, 2013

(54) EICOSAPENTAENOIC ACID CONCENTRATE

(71) Applicants: Shu-Chien Liang, Newark, DE (US); Robert D. Orlandi, Landenberg, PA (US)

(72) Inventors: Shu-Chien Liang, Newark, DE (US); Robert D. Orlandi, Landenberg, PA (US)

(73) Assignee: E I DU PONT DE NEMOURS AND COMPANY, Wilmington, DE (US)

(21) Appl. No.: 13/370,899

(22) Filed: Feb. 10, 2012

Related U.S. Application Data
(60) Provisional application No. 61/441,854, filed on Feb. 11, 2011.
(60) Provisional application No. 61/487,019, filed on May 17, 2011.

(51) Int. Cl. A61K 31/557; A61P 3/06; A61P 9/00; A61P 29/00; A61P 35/00; A61P 13/12; A61P 11/00; A61P 3/10; A61P 3/04; A61P 1/04; A61P 1/00; A61P 17/02; A61P 19/02; A61P 25/00; A61P 25/18; A61P 3/02; A61P 7/02

(52) U.S. Cl. 514/560

(57) ABSTRACT

An omega-3 oil concentrate comprising at least 70 weight percent of eicosapentaenoic acid [“EPA”; cis-5,8,11,14,17-eicosapentaenoic acid; omega-3], measured as a weight percent of oil, and substantially free of docosahexaenoic acid, said concentrate obtained from a microbial oil having 30 to 70 weight percent of eicosapentaenoic acid, measured as a weight percent of total fatty acids, and substantially free of docosahexaenoic acid and wherein said microbial oil is obtained from a microorganism that accumulates in excess of 25% of its dry cell weight as oil. Also disclosed are methods of making such eicosapentaenoic acid concentrates.

This application claims the benefit of U.S. Provisional Application No. 61/441,854, filed Feb. 11, 2011, and U.S. Provisional Application No. 61/487,019, filed May 17, 2011, which are hereby incorporated by reference in their entirety.

### FIELD OF THE INVENTION

This invention pertains to an omega-3 oil concentrate comprising the long-chain polyunsaturated fatty acid cis-5,8,11,14,17-eicosapentaenoic acid [“EPA”] and, more particularly, to an EPA concentrate comprising at least 70 weight percent of EPA, measured as a weight percent of oil, and substantially free of cis-4,7,10,13,16,19-docosahexaenoic acid [“DHA”].

### BACKGROUND OF THE INVENTION

Health benefits derived from supplementation of the diet with omega-3 fatty acids, such as alpha-linolenic acid [“ALA”](18:3), stearidonic acid [“STA”](18:4), eicosatetraenoic acid [“ETrA”](20:3), eicosatrienoic acid [“ETA”](20:4), eicosapentaenoic acid [“EPA”](20:5), docosapentaenoic acid [“DPA”](22:5) and docosahexaenoic acid [“DHA”](22:6), are well recognized and supported by numerous clinical studies and other published public and patent literature. For example, omega-3 fatty acids have been found to have beneficial effects on the risk factors for cardiovascular diseases, especially mild hypertension, hypertriglyceridemia and on coagulation factor VII phospholipid complex activity.

With respect to eicosapentaenoic acid [“EPA”; cis-5,8,11,14,17-eicosapentaenoic acid; omega-3], the clinical and pharmaceutical value of this particular fatty acid is well established (U.S. Pat. Appl. Publications No. 2009-0093543-A1 and No. 2010-0317072-A1). EPA is an important intermediate in the biosynthesis of biologically active prostaglandin. Additionally, the following pharmacological actions of EPA are known: 1) platelet coagulation inhibitory action (thrombolytic action); 2) blood neutral fat-lowering action; 3) blood very-low-density lipoprotein [“VLDL”]-cholesterol and low-density lipoprotein [“LDL”]-cholesterol lowering action and blood high-density lipoprotein [“HDL”]-cholesterol (anti-arterial sclerosis action) raising action; 4) blood viscosity-lowering action; 5) blood pressure lowering action; 6) anti-inflammatory action; and, 7) anti-tumor action. As such, EPA provides a natural approach to lower blood cholesterol and triglycerides.

Increased intake of EPA has been shown to be beneficial or have a positive effect in coronary heart disease, high blood pressure, inflammatory disorders (e.g., rheumatoid arthritis), lung and kidney diseases, Type II diabetes, obesity, ulcerative colitis, Crohn's disease, anorexia nervosa, burns, osteoarthritis, osteoporosis, attention deficit/hyperactivity disorder, and early stages of colorectal cancer. See, for example, the review of McColl, J., NutraCos, 2(4):35-40 (2003), and Sinclair, A., et al. In Healthful Lipids, C. C. Akoh and O.-M. Lai, Eds, AOCS: Champaign, Ill., 2005, Chapter 16. Recent findings have also confirmed the use of EPA in the treatment of mental disorders, such as schizophrenia (U.S. Pat. No. 6,331,568 and U.S. Pat. No. 6,624,195). As a result, EPA is used in products relating to functional foods (nutraceuticals), medical foods, infant nutrition, bulk nutrition, cosmetics and animal health.

Despite abundant research in the area of omega-3 fatty acids, however, many past studies have failed to recognize that individual long-chain omega-3 fatty acids (e.g., EPA and DHA) are metabolically and functionally distinct from one another, and thus each may have specific physiological functions and biological activities.

This lack of mechanistic clarity is largely a consequence of the use of fish oils which contain a variable mixture of omega-3 fatty acids, as opposed to using pure EPA or pure DHA in clinical studies [the fatty acid composition of oils from menhaden, cod liver, sardines and anchovies, for example, comprise oils having a ratio of EPA:DHA of approximately 0.9:1 to 1.6:1 (based on data within The Lipid Handbook, 2^{nd} ed.; F. D. Gunstone, J. L. Harwood and F. B. Padley, Eds; Chapman and Hall, 1994)]. Additionally, fish oils also contain significant amounts of cholesterol and thus daily consumption of fish oils may increase cholesterol uptake, thereby counteracting any reduction of blood lipid levels.

There is a pharmaceutical composition sold under the trademark OMACOR® and now known as LOVAZA™[U.S. Pat. No. 5,502,077, U.S. Pat. No. 5,656,667 and U.S. Pat. No. 5,698,594](Pronova Biocare A.S., Lysaker, Norway) that is a combination of ethyl esters of DHA and EPA. Each capsule contains approximately 430 mg/g-495 mg/g EPA and 347 mg/g-403 mg/g DHA with 90% (w/w) [“weight by weight”] total omega-3 fatty acids.

Omega-3 fatty acids at high doses are known to have significant triglyceride lowering properties. Four capsules per day of a concentrated formulation of omega-3 ethyl esters has been approved in the United States by the Food and Drug Administration for triglyceride lowering in patients with fasting triglycerides over 500 mg/dl. Each of these one gram capsules contains 465 mg of EPA and 375 mg of DHA, for a total daily dose of 1,860 mg of EPA and 1,500 mg of DHA within the 4 capsules. This formulation at this dose has been reported to decrease triglyceride levels by 29.5% and raise HDL cholesterol by 3.4% versus placebo (both p<0.05) in subjects with triglyceride levels between 200 and 500 mg/dl on 40 mg simvastatin per day (Davidson, M. H. et al., Clin. Ther., 29:1354-1367 (2007)). Even greater triglyceride reductions are observed in subjects with triglyceride levels over 500 mg/dl. It has also been documented that DHA at doses of approximately 1200 mg/day will significantly lower triglyceride levels by about 25% (Davidson, M. H. et al., J. Am. Coll. Nutr., 16(3):236-243 (1997); Berson, E. L. et al., Arch. Opthalmol., 122:1297-1305 (2004)).

Although both LOVAZA™ and pure EPA have been shown to lower triglycerides, LOVAZA™ has been associated with the unfavorable consequence of increased LDL-cholesterol while supplementation with pure EPA does not result in this effect. It is believed that this difference may be due to the presence of DHA in LOVAZA™. Consequently, since it appears that cardiovascular benefits can be achieved using EPA alone, an omega-3 therapy comprising EPA and substantially no DHA is preferable.

Few studies have been performed with substantially pure EPA and separately with substantially pure DHA, to enable differentiation of the pharmacological effects of each individual fatty acid. One exception is the Japanese EPA Lipid Intervention Study [“JELIS”], which involved a large-scale randomized controlled trial using >98% purified EPA-ethyl esters [EPA-EE”](Mochida Pharmaceutical, Ltd.) in combination with a statin (Yokoyama, M. and H. Origasa, Amer. Heart J., 146:613-620 (2003); Yokoyama, M. et al., Lancet, 369:1090-1098 (2007)). It was found that cardiovascular events in patients receiving EPA plus statin decreased by 19% with respect to those patients receiving statin alone. This provides strong support that EPA, per se, is cardioprotective; similar studies using DHA have not been reported.

Several citations describe use of highly purified EPA compositions for various pharmaceutical purposes. For example: i) GB Patent Application No. 1,604,554, published on Dec. 9, 1981, describes the use of EPA in treating thrombo-embolic conditions wherein at least 50% by weight of the fatty acid composition should be EPA; ii) U.S. Pat. Appl. Pub. No. 2008-0200547 discloses a pharmaceutical preparation comprising at least 90% EPA and preferably 95% EPA, and less than 5%, more preferably less than 3%, in the form of DHA; iii) U.S. Pat. No. 7,498,359 (Mochida Pharmaceutical, Ltd.) describes administration of a high purity EPA-EE [sold under the trademark Epadel® and Epadel® S in Japan] that is useful for reducing recurrence of stroke when administered in combination with a 3-hydroxy-3-methylglutaryl coenzyme A [“HMG-CoA”] reductase inhibitor; iv) Intl. Appl. Pub. No. WO 2010/093634 A1, published on Aug. 19, 2010, describes the use of EPA-EE for treating hypertriglyceridemia; v) Intl. Appl. Pub. No. WO 2010/147994 A1, published on Dec. 23, 2010, describes methods of lowering triglycerides in subjects on statin therapy, by administration of ultra-pure EPA comprising at least 96% by weight; and, yl) U.S. Pat. Pub. No. 2011-0178105-A1 describes methods of maintaining or lowering lipoprotein-associated phospholipase A₂ [“Lp-PLA₂”] levels, stabilizing rupture prone-atherosclerotic lesions, decreasing the Inflammatory Index and increasing Total Omega-3 Score™ in humans, by administration of EPA.

Since EPA and other long-chain polyunsatured fatty acids have very similar physical properties (e.g., similar vapor pressure, solubility, and adsorption characteristics), separation and purification of EPA to high purity is complex. Various methods for enriching EPA content of a fatty acid mixture from various natural sources are known (e.g., low temperature crystallization, urea adduct formation, fractional distillation, high pressure liquid chromatography, treatment with silver salt, supercritical carbon dioxide [“CO₂”]chromatography, supercritical CO₂ fractionation with counter-current column, simulated moving bed chromatography, actual moving bed chromatography, etc. and combinations thereof).

For example, downstream processing methods to enrich EPA from several types of red and green algae and marine diatoms have been described, as set forth below.
(i) Cohen et al. (J. Amer. Chem. Soc., 68(1):16-19 (1991)) describe purification from the red microalga Porphyridium cruentum.
(ii) Medina et al. (Biotechnology Advances, 16(3):517-580 (1998)) provide a review of means to purify polyunsaturated fatty acids [“PUFAs”], e.g., EPA, from microalgae.
(iii) U.S. Pat. No. 4,615,839 discloses processes for extraction of marine Chlorella, wherein the resulting lipid composition was subjected to solvent fractionation to remove neutral fats, thereby providing a polar lipid composition. The polar lipid composition was subjected to hydrolysis to liberate fatty acids which were recovered, thereby providing a fatty acid composition with at least 60% by weight of EPA. Urea treatment of this fatty acid composition enriched the EPA content to 93.0%. DHA content was not disclosed.
(iv) U.S. Pat. Appl. Pub. No. 2010/0069492 describes the recovery of an EPA composition from enzyme-hydrolyzed lipids of the diatom Nitzschia laevis, whereby the fatty acid content comprised 50-60% EPA, less than 5.5% arachidonic acid [“ARA”, omega-6] and substantially no DHA. It was suggested, but not exemplified, that the EPA could be further purified to between 95% and 99%, less than 1% of ARA and less than 0.1% DHA.

Similarly, numerous references describe purification of EPA from fish oils (or from mixtures of fatty acid ethyl esters obtained from fish oils). For example:
(i) Beebe et al. (J. Chromatography, 459:369-378 (1988)) describe preparative scale high performance liquid chromatography [“HPLC”] of omega-3 PUFA esters.
(ii) U.S. Pat. No. 4,377,526 describes transesterification to the ethyl ester, followed by urea treatment and fractional distillation. The resulting product was reported to comprise 92.9% EPA and 2.0% DHA.
(iii) U.S. Pat. No. 5,215,630 discloses fractional distillation at low pressure using a system of at least three distillation columns. The product comprised 99.9% fatty acids having chain lengths of C₂₀ [“C20”], wherein 88% of the C20 fraction was EPA. Urea treatment of the C20 fraction increased the EPA content to 93%.
(iv) U.S. Pat. No. 5,719,302 discloses a purification process including a step of (a) treating the fatty acid ethyl ester mixture by either (1) stationary bed chromatography or (2) multistage countercurrent column fractionation in which a solvent is a fluid at supercritical pressure, and recovering at least one PUFA-enriched fraction. The process also includes a step of (b) subjecting the fraction recovered in the treating step to further fractionation by simulated continuous countercurrent moving bed chromatography and recovering at least one fraction containing the purified PUFA or the PUFA mixture. Fractions with 88% EPA and 0.8% DHA, and >93% EPA (DHA content was not disclosed) were obtained.
(v) U.S. Pat. No. 5,840,944 discloses precision distillation under high vacuum to produce a concentrated mixture of esters comprising 99.9% C20, of which 82.77% was EPA. Subjecting the EPA enriched mixture to high speed liquid chromatography yielded an oil with 99.5% EPA (DHA was not specifically reported but total acids >C20 was 0.30%).
(vi) Japan Unexamined Patent Publication Heisei 9-310089 (JP1997310089) discloses purification of fish oil ethyl ester by supercritical CO₂ extraction with multiple extraction columns. A product comprising 90.8% EPA and 0.35% DHA was obtained from a fatty acid ester starting mixture comprising 41.1% EPA and 17.3% DHA.
(vii) Japan Unexamined Patent Publication Heisei 9-302380 (JP1997302380) discloses the fractionation of fatty acid esters derived from fish oil by a three column distillation process to produce a main fraction with 82% EPA. The main fraction was further purified by treatment with silver salt to obtain 98.5% EPA-EE.
(viii) Intl. Appl. Pub. No. WO 01/36369 A1 discloses a method for preparing EPA-EE with at least 95% purity by column chromatography using supercritical CO₂ as the mobile phase starting from a mixture of fatty acid esters having an EPA-EE content of 50% and a maximum content of 1.2% of arachidonic acid.
(ix) Int'l. Appl. Pub. No. WO 2011/080503 A2 discloses a chromatographic separation process for recovering a PUFA product, from a feed mixture, comprising introducing the feed mixture to a simulated or actual moving bed chromatography apparatus having a plurality of linked chromatography columns containing, as eluent, an aqueous alcohol, wherein the apparatus has a plurality of zones comprising at least a first zone and second zone, each zone having an extract stream and a raffinate stream from which liquid can be collected from said plurality of linked chromatography columns, and wherein (a) a raffinate stream containing the PUFA product together with more polar components is collected from a column in the first zone and introduced to a nonadjacent column in the second zone, and/or (b) an extract stream containing the PUFA product together with less polar components is collected from a column in the second zone and introduced to a nonadjacent column in the first zone, said PUFA product being separated from different components of the feed mixture in each zone. Various fish oil derived feedstocks were purified to produce 85 to greater than 98% EPA EE. Although Int'l. Appl. Pub. No. WO 2001/080503 A2 demonstrated processes to recover EPA and DHA in high purity from fish oils, the disclosure also states that suitable feed mixtures for fractionating may be obtained from “synthetic sources including oils obtained from genetically modified plants, animals and microorganisms including yeasts”. Further, “genetically modified yeast is particularly suitable when the desired PUFA product is EPA”.

Finally, U.S. Pat. No. 5,189,189 discloses the enrichment of a fatty acid mixture containing 60% EPA by treatment with silver salt, resulting in a product comprising 96.0% EPA. Repeating the silver salt treatment further increased the EPA content to 98.5%. Neither the identity of the other constituent fatty acids nor the source of the 60% EPA starting mixture was disclosed.

One concern that arises when purifying EPA from natural marine sources (e.g., fish, algae) is the co-presence of relatively high concentrations of environmental pollutants within these organisms, as a result of bioaccumulation. These environmental pollutants are toxic components, such as polychlorinated biphenyls [“PCBs”](CAS No. 1336-36-3), brominated flame retardants, pesticides (e.g., toxaphenes and dichlorodiphenyltrichloroethane [“DDT”] and its metabolites), and other organic compounds found in the sea environment that are potentially harmful and/or toxic. U.S. Pat. No. 7,732,488 discloses a process for decreasing the amount of environmental pollutants in a mixture comprising a fat or an oil such as fish oil.

Disregarding concerns of pollutants, however, the environmental impact of purifying EPA from natural marine sources must also be considered in light of global over-fishing. Currently, it is estimated that feed compositions for aquaculture use about 87% of the global supply of fish oil as a lipid source. Since annual fish oil production has not increased beyond 1.5 million tons per year, industries—including the rapidly growing one of aquaculture—cannot continue to rely on finite stocks of marine pelagic fish as a supply of fish oil. Many organizations recognize the limitations noted above with respect to fish oil availability and sustainability and are seeking alternative ingredients that will reduce dependence on fish oil, while maintaining the important benefits of this ingredient in the products and industries where it is used. The production of EPA concentrates for human consumption from a sustainable source, versus marine sources, would thus have a positive environmental impact.

In view of the mounting benefits and increasing demand for EPA as a therapeutic agent, a need exists for improved sources of EPA, as well as preparative methods to enrich EPA to appropriate pharmaceutical concentrations. Preferably, concentrated EPA oils intended for human consumption will have substantially no DHA and substantially no environmental pollutants.

### SUMMARY OF THE INVENTION

In one embodiment, the present invention pertains to an eicosapentaenoic acid concentrate comprising at least 70 weight percent of eicosapentaenoic acid [“EPA”], measured as a weight percent of oil, and substantially free of docosahexaenoic acid [“DHA”], said concentrate obtained from a microbial oil comprising 30 to 70 weight percent of eicosapentaenoic acid, measured as a weight percent of total fatty acids, and substantially free of docosahexaenoic acid, wherein said microbial oil is obtained from a microorganism that accumulates in excess of 25% of its dry cell weight as oil.

In a second embodiment, the microbial oil:
a) comprises from about 1 to about 25 weight percent linoleic acid, measured as a weight percent of total fatty acids; and,
b) has a ratio of at least 1.2 of eicosapentaenoic acid, measured as a weight percent of total fatty acids, to linoleic acid, measured as a weight percent of total fatty acids.

In a third embodiment, the microbial oil is a microbial oil obtained from microbial biomass of recombinant Yarrowia cells, engineered for the production of eicosapentaenoic acid.

In a fourth embodiment, the invention concerns a pharmaceutical product comprising the eicosapentaenoic acid concentrate of the invention.

In a fifth embodiment, the invention concerns a method for making an eicosapentaenoic acid concentrate comprising at least 70 weight percent of eicosapentaenoic acid, measured as a weight percent of oil, and substantially free of docosahexaenoic acid, said method comprising:
a) transesterifying a microbial oil comprising 30 to 70 weight percent of eicosapentaenoic acid, measured as a weight percent of total fatty acids, and substantially free of DHA, wherein said microbial oil is obtained from a microorganism that accumulates in excess of 25% of its dry cell weight as oil; and,
b) enriching the transesterified oil of step (a) to obtain an eicosapentaenoic acid concentrate comprising at least 70 weight percent of eicosapentaenoic acid, measured as a weight percent of oil, and substantially free of docosahexaenoic acid.
   The transesterified oil of step (b) may be enriched by a process selected from the group consisting of: urea adduct formation, liquid chromatography, supercritical fluid chromatography, fractional distillation, simulated moving bed chromatography, actual moving bed chromatography and combinations thereof.

In a sixth embodiment, the method of the invention concerns use of a microbial oil having a ratio of at least 1.2 of eicosapentaenoic acid, measured as a weight percent of total fatty acids, to linoleic acid, measured as a weight percent of total fatty acids. Furthermore, the microbial oil can be a microbial oil obtained from microbial biomass of recombinant Yarrowia cells, engineered for the production of eicosapentaenoic acid.

In a seventh embodiment, the eicosapentaenoic acid concentrate of the invention is substantially free of environmental pollutants.

In an eighth embodiment, the invention concerns the use of a microbial oil having 30 to 70 weight percent of eicosapentaenoic acid, measured as a weight percent of total fatty acids, and substantially free of docosahexaenoic acid, to make an eicosapentaenoic acid concentrate comprising at least 70 weight percent of eicosapentaenoic acid, measured as a weight percent of oil, and substantially free of docosahexaenoic acid,

wherein said microbial oil is obtained from a microorganism that accumulates in excess of about 25% of its dry cell weight as oil.

In a ninth embodiment, the microbial oil in any of the above embodiments is non-concentrated.

In a tenth embodiment, the microbial oil in any of the above embodiments is substantially free of a fatty acid selected from the group consisting of nonadecapentaenoic acid and heneicosapentaenoic acid.

In an eleventh embodiment, the eicosapentaenoic acid concentrate of the invention is substantially free of a fatty acid selected from the group consisting of nonadecapentaenoic acid and heneicosapentaenoic acid.

### BIOLOGICAL DEPOSITS

The following biological materials have been deposited with the American Type Culture Collection (ATCC), 10801 University Boulevard, Manassas, Va. 20110-2209, and bear the following designations, accession numbers and dates of deposit.

| Biological Material | Accession No. | Date of Deposit |
|---|---|---|
| Yarrowia lipolytica Y8412 | ATCC PTA-10026 | May 14, 2009 |
| Yarrowia lipolytica Y8259 | ATCC PTA-10027 | May 14, 2009 |

The biological materials listed above were deposited under the terms of the Budapest Treaty on the International Recognition of the Deposit of Microorganisms for the Purposes of Patent Procedure. The listed deposits will be maintained in the indicated international depository for at least 30 years and will be made available to the public upon the grant of a patent disclosing it. The availability of a deposit does not constitute a license to practice the subject invention in derogation of patent rights granted by government action.

Yarrowia lipolytica Y9502 was derived from Y. lipolytica Y8412, according to the methodology described in U.S. Pat. Appl. Pub. No. 2010-0317072-A1. Similarly, Yarrowia lipolytica Y8672 was derived from Y. lipolytica Y8259, according to the methodology described in U.S. Pat. Appl. Pub. No. 2010-0317072-A1.

### BRIEF DESCRIPTION OF THE DRAWINGS AND SEQUENCE LISTING

FIG. 1 provides an overview of the processes of the invention, in the form of a flowchart. Specifically, a microbial fermentation produces untreated microbial biomass, which may optionally be mechanically processed. Oil extraction of the untreated microbial biomass results in residual biomass and extracted oil. The extracted oil can be directly transesterified and enriched to produce an EPA concentrate comprising at least 70 weight percent [“wt %”] EPA, measured as a wt % of oil, and substantially free of DHA; or, the extracted oil can first be either: i) purified via degumming, refining, bleaching, deodorization, etc.; or, ii) distilled using short path distillation (SPD).

FIG. 2 diagrams the development of various Yarrowia lipolytica strains derived from Yarrowia lipolytica ATCC #20362.

FIG. 3 provides plasmid maps for the following: (A) pZKUM; and, (B) pZKL3-9DP9N.

The following sequences comply with 37 C.F.R. §1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NOs:1-8 are open reading frames encoding genes, proteins (or portions thereof), or plasmids, as identified in Table 1.

TABLE 1
Summary Of Nucleic Acid And Protein SEQ ID Numbers

| | | Protein |
|---|---|---|
| | Nucleic acid | SEQ |
| Description | SEQ ID NO. | ID NO. |
| Plasmid pZKUM | 1 | — |
| | (4313 bp) | |
| Plasmid pZKL3-9DP9N | 2 | — |
| | (13,565 bp) | |
| Synthetic mutant delta-9 elongase, derived | 3 | 4 |
| from Euglena gracilis (“EgD9eS-L35G”) | (777 bp) | (258 AA) |
| Yarrowia lipolytica delta-9 desaturase gene | 5 | 6 |
| (Gen Bank Accession No. XM_501496) | (1449 bp) | (482 AA) |
| Yarrowia lipolytica choline-phosphate | 7 | 8 |
| cytidylyl-transferase gene (GenBank | (1101 bp) | (366 AA) |
| Accession No. XM_502978) | | |

### DETAILED DESCRIPTION OF THE INVENTION

All patents, patent applications, and publications cited herein are incorporated by reference in their entirety.

The following definitions are provided.

“Eicosapentaenoic acid” is abbreviated as “EPA”.

“American Type Culture Collection” is abbreviated as “ATCC”.

“Polyunsaturated fatty acid(s)” is abbreviated as “PUFA(s)”.

“Triacylglycerols” are abbreviated as “TAGs”.

“Total fatty acids” are abbreviated as “TFAs”.

“Fatty acid methyl esters” are abbreviated as “FAMEs”.

“Ethyl ester” is abbreviated as “EE”.

“Dry cell weight” is abbreviated as “DCW”.

“Weight percent” is abbreviated as “wt %”.

As used herein the term “invention” or “present invention” is intended to refer to all aspects and embodiments of the invention as described in the claims and specification herein and should not be read so as to be limited to any particular embodiment or aspect.

The term “pharmaceutical” as used herein means a compound or substance which, if sold in the United States, would be controlled by Section 503 or 505 of the Federal Food, Drug and Cosmetic Act.

The term “eicosapentaenoic acid concentrate” or “EPA concentrate” refers to an omega-3 oil comprising at least 70 wt % of EPA, measured as a wt % of oil, and substantially free of DHA. The oil concentrate is obtained from a microbial oil comprising 30 to 70 wt % of EPA, measured as a wt % of total fatty acids, and substantially free of DHA, wherein said microbial oil is obtained from a microorganism that accumulates in excess of 25% of its dry cell weight as oil, as will be elaborated hereinbelow. The at least 70 wt % of EPA will be in the form of free fatty acids, triglycerides (e.g., TAGs), esters, and combinations thereof. The esters are most preferably in the form of ethyl esters.

The term “microbial biomass” refers to microbial cellular material from a microbial fermentation, the cellular material comprising EPA. The microbial biomass may be in the form of whole cells, whole cell lysates, homogenized cells, partially hydrolyzed cellular material, and/or partially purified cellular material (e.g., microbially produced oil). In preferred embodiments, the microbial biomass refers to spent or used microbial cellular material from the fermentation of a production host producing EPA in commercially significant amounts, such as recombinantly engineered strains of the oleaginous yeast, Yarrowia lipolytica.

The term “untreated microbial biomass” refers to microbial biomass prior to extraction with a solvent. Optionally, untreated microbial biomass may be subjected to mechanical processing (e.g., by drying the biomass, disrupting the biomass, or a combination of these) prior to extraction with a solvent.

As used herein the term “residual biomass” refers to microbial cellular material from a microbial fermentation comprising EPA, which has been extracted at least once with a solvent (e.g., an inorganic or organic solvent).

The term “oil” refers to a lipid substance that is liquid at 25° C. and usually polyunsaturated. In oleaginous organisms, oil constitutes a major part of the total lipid and is composed primarily of triacylglycerols [“TAGs”] but may also contain other neutral lipids, phospholipids and free fatty acids. After purification or enrichment of a specific fatty acid in such an oil, the oil can exist in various chemical forms (e.g., in the form of triacylglycerols, alkyl esters, salts or free fatty acids). The fatty acid composition in the oil and the fatty acid composition of the total lipid are generally similar; thus, an increase or decrease in the concentration of PUFAs in the total lipid will correspond with an increase or decrease in the concentration of PUFAs in the oil, and vice versa.

The term “extracted oil” or “crude oil” (as the terms can be used interchangeably herein) refers to an oil that has been separated from other cellular materials, such as the organism in which the oil was synthesized. Extracted oils are obtained through a wide variety of methods, the simplest of which involves physical means alone. For example, mechanical crushing using various press configurations (e.g., screw, expeller, piston, bead beaters, etc.) can separate oil from cellular materials. Alternately, oil extraction can occur via treatment with various organic solvents (e.g., hexane), enzymatic extraction, osmotic shock, ultrasonic extraction, supercritical fluid extraction (e.g., CO₂ extraction), saponification and combinations of these methods. Further purification or concentration of an extracted oil is optional.

The term “microbial oil” is a generic term and, thus, may refer to either a non-concentrated microbial oil or a concentrated microbial oil, as further defined hereinbelow.

The term “non-concentrated microbial oil” means that the microbial oil obtained via extraction has not been substantially enriched in one or more fatty acids. In other words, the fatty acid composition of the “non-concentrated microbial oil” which may have been separated from the cellular materials of the microorganism is substantially similar to the fatty acid composition of the oil as produced by the microorganism. Thus, the non-concentrated microbial oils utilized herein comprise at least 30 to 70 EPA % TFAs since the microorganisms producing these oils have a fatty acid composition comprising at least 30 to 70 EPA % TFAs. The non-concentrated microbial oil may be non-concentrated extracted oil or non-concentrated purified oil.

As those skilled in the art will appreciate, it is possible to start with a microbial oil having less than 30 EPA % TFAs and process it so that the microbial oil comprises a sufficient amount of EPA % TFAs to use it in making the EPA concentrate of the invention.

The term “purified oil” refers to a microbial oil having reduced concentrations of impurities, such as phospholipids, trace metals, free fatty acids, color compounds, minor oxidation products, volatile and/or odorous compounds, and sterols (e.g., ergosterol, brassicasterol, stigmasterol, cholesterol), as compared to the concentrations of impurities in the extracted oil. Purification processes do not typically concentrate or enrich the microbial oil, such that a particular fatty acid(s) is substantially enriched, and thus purified oil is most often non-concentrated.

The term “distilling” refers to a method of separating mixtures based on differences in their volatilities in a boiling liquid mixture. Distillation is a unit operation, or a physical separation process.

The term “short path distillation” [“SPD”] refers to a separation method operating under an extremely high vacuum, in which the SPD device is equipped with an internal condenser in close proximity to the evaporator, such that volatile compounds from the material to be distilled after evaporation travel only a short distance to the condensing surface. As a result, there is minimal thermal degradation from this separation method.

The term “SPD-purified oil” refers to a microbial oil containing a triacylglycerol-fraction comprising one or more PUFAs, said oil having undergone a process of distillation at least once under SPD conditions. The distillation process reduces the amount of sterol in the SPD purified oil, as compared to the sterol content in the oil prior to SPD. Although SPD can concentrate ethyl esters, methyl esters and free fatty acids, the process does not typically concentrate TAGs (e.g., unless operated at extremely high temperatures which then leads to decomposition of TAGs). Since the majority of PUFAs in extracted oil are in the form of TAGs, and the SPD process does not typically concentrate TAGs such that a particular fatty acid(s) is substantially enriched, the SPD-purified oil is considered to be non-concentrated most often for the purposes described herein.

The term “transesterification” refers to a chemical reaction, catalyzed by an acid or base catalyst, in which an ester of a fatty acid is converted to a different ester of the fatty acid.

The term “enrichment” refers to a process to increase the concentration of one or more fatty acids in a microbial oil, relative to the concentration of the one or more fatty acids in the non-concentrated microbial oil. Thus, as discussed herein, a microbial oil comprising 30 to 70 wt % of EPA, measured as a wt % of TFAs, is enriched or concentrated to produce an EPA concentrate comprising at least 70 wt % of EPA, measured as a wt % of oil.

The term “fatty acids” refers to long chain aliphatic acids (alkanoic acids) of varying chain lengths, from about C₁₂ to C₂₂ (or C12 to C22, wherein the number refers to the total number of carbon [“C”] atoms in the chain) although both longer and shorter chain-length acids are known. The predominant chain lengths are between C₁₆ and C₂₂. The structure of a fatty acid is represented by a simple notation system of “X:Y”, where X is the total number of carbon [“C”] atoms in the particular fatty acid and Y is the number of double bonds. Additional details concerning the differentiation between “saturated fatty acids” versus “unsaturated fatty acids”, “monounsaturated fatty acids” versus “polyunsaturated fatty acids” [“PUFAs”], and “omega-6 fatty acids” [“ω-6” or “n-6”] versus “omega-3 fatty acids” [“ω-3” or “n-3”] are provided in U.S. Pat. No. 7,238,482, which is hereby incorporated herein by reference.

Nomenclature used to describe PUFAs herein is given in Table 2. In the column titled “Shorthand Notation”, the omega-reference system is used to indicate the number of carbons, the number of double bonds and the position of the double bond closest to the omega carbon, counting from the omega carbon, which is numbered 1 for this purpose. The remainder of the Table summarizes the common names of omega-3 and omega-6 fatty acids and their precursors, the abbreviations that will be used throughout the specification and the chemical name of each compound.

TABLE 2
Nomenclature of Polyunsaturated Fatty Acids And Precursors

| | | | Shorthand |
|---|---|---|---|
| Common Name | Abbreviation | Chemical Name | Notation |
| Myristic | — | Tetradecanoic | 14:0 |
| Palmitic | Palmitate | Hexadecanoic | 16:0 |
| Palmitoleic | — | 9-hexadecenoic | 16:1 |
| Stearic | — | Octadecanoic | 18:0 |
| Oleic | — | cis-9-octadecenoic | 18:1 |
| Linoleic | LA | cis-9,12- | 18:2 ω-6 |
| | | octadecadienoic | |
| Gammã-Linolenic | GLA | cis-6,9,12- | 18:3 ω-6 |
| | | octadecatrienoic | |
| Eicosadienoic | EDA | cis-11,14- | 20:2 ω-6 |
| | | eicosadienoic | |
| Dihomo-Gamma | DGLA | cis-8,11,14- | 20:3 ω-6 |
| Linolenic | | eicosatrienoic | |
| Arachidonic | ARA | cis-5,8,11,14- | 20:4 ω-6 |
| | | eicosatetraenoic | |
| Alphã-Linolenic | ALA | cis-9,12,15- | 18:3 ω-3 |
| | | octadecatrienoic | |
| Stearidonic | STA | cis-6,9,12,15- | 18:4 ω-3 |
| | | octadecatetraenoic | |
| Nonadecapentaenoic | NDPA | cis-5,8,11,14,17- | 19:5 ω-2 |
| | | nonadecapentaenoic | |
| Eicosatrienoic | ETrA | cis-11,14,17- | 20:3 ω-3 |
| | | eicosatrienoic | |
| Eicosatetraenoic | ETA | cis-8,11,14,17- | 20:4 ω-3 |
| | | eicosatetraenoic | |
| Eicosapentaenoic | EPA | cis-5,8,11,14,17- | 20:5 ω-3 |
| | | eicosapentaenoic | |
| Heneicosapentaenoic | HPA | cis-6,9,12,15,18- | 21:5 ω-3 |
| | | Heneicosapentaenoic | |
| Docosatetraenoic | DTA | cis-7,10,13,16- | 22:4 ω-6 |
| | | docosatetraenoic | |
| Docosapentaenoic | DPAn-6 | cis-4,7,10,13,16- | 22:5 ω-6 |
| | | docosapentaenoic | |
| Docosapentaenoic | DPA | cis-7,10,13,16,19- | 22:5 ω-3 |
| | | docosapentaenoic | |
| Docosahexaenoic | DHA | cis-4,7,10,13,16,19- | 22:6 ω-3 |
| | | docosahexaenoic | |

Thus, the term “eicosapentaenoic acid” [“EPA”] is the common name for cis-5,8,11,14,17-eicosapentaenoic acid. This fatty acid is a 20:5 omega-3 fatty acid. The term EPA, as used in the present disclosure, will refer to the acid or derivatives of the acid (e.g., glycerides, esters, phospholipids, amides, lactones, salts or the like), unless specifically mentioned otherwise. For example, “EPA-EE” will specifically refer to EPA ethyl ester.

“Docosahexaenoic acid” [“DHA”] is the common name for cis-4,7,10,13,16,19-docosahexaenoic acid; this fatty acid is a 22:6 omega-3 fatty acid. The term DHA as used in the present disclosure will refer to the acid or derivatives of the acid (e.g., glycerides, esters, phospholipids, amides, lactones, salts or the like), unless specifically mentioned otherwise.

“Nonadecapentaenoic acid” [“NDPA”] is the common name for cis-5,8,11,14,17-nonadecapentaenoic acid; this fatty acid is a 19:5 omega-2 fatty acid. “Heneicosapentaenoic acid” [“HPA”] is the common name for cis-6,9,12,15,18-heneicosapentaenoic acid; this fatty acid is a 21:5 omega-3 fatty acid. Both of these fatty acids are commonly found in fish oils. Concentrated EPA produced from fish oils will often contain these fatty acids as impurities in the final EPA composition (see, e.g., U.S. Pat. Appl. Pub. No. 2010-0278879 and Intl. Appl. Pub. No. WO 2010/147994 A1). The terms NDPA and HPA as used in the present disclosure will refer to the respective acid or derivatives of the acid (e.g., glycerides, esters, phospholipids, amides, lactones, salts or the like), unless specifically mentioned otherwise.

The term “‘lipids” refer to any fat-soluble (i.e., lipophilic), naturally-occurring molecule. A general overview of lipids is provided in U.S. Pat. Appl. Pub. No. 2009-0093543-A1 (see Table 2 therein).

The term “triacylglycerols” [“TAGs”] refers to neutral lipids composed of three fatty acyl residues esterified to a glycerol molecule. TAGs can contain long chain PUFAs and saturated fatty acids, as well as shorter chain saturated and unsaturated fatty acids. In living organisms, TAGs are the primary storage units for fatty acids since the glycerol backbone helps to stabilize PUFA molecules for storage or during transport. In contrast, free fatty acids are rapidly oxidized.

“Fatty acid ethyl esters” [“FAEEs”] refer to a chemical form of lipids that are generally synthetically derived by reacting free fatty acids or their derivatives with ethanol, in a process of esterification or transesterification.

The term “total fatty acids” [“TFAs”] herein refer to the sum of all cellular fatty acids that can be derivitized to fatty acid methyl esters [“FAMEs”] by the base transesterification method (as known in the art) in a given sample, which may be the microbial biomass or oil, for example. Thus, TFAs include fatty acids from neutral lipid fractions (including diacylglycerols, monoacylglycerols and TAGs) and from polar lipid fractions (including, e.g., the phosphatidylcholine and the phosphatidylethanolamine fractions) but not free fatty acids.

The term “total lipid content” of cells is a measure of TFAs as a percent of the dry cell weight [“DCW”], although total lipid content can be approximated as a measure of FAMEs as a percent of the DCW [“FAMEs % DCW”]. Thus, total lipid content [“TFAs % DCW”] is equivalent to, e.g., milligrams of total fatty acids per 100 milligrams of DCW.

The concentration of a fatty acid in the total lipid is expressed herein as a weight percent of TFAs [“% TFAs”], e.g., milligrams of the given fatty acid per 100 milligrams of TFAs. This unit of measurement is used to describe the concentration of, e.g., EPA, in microbial cells and in microbial oil.

The concentration of a fatty acid ester (and/or fatty acid and/or triglyceride, respectively) in the oil is expressed as a weight percent of oil [“% oil”], e.g. milligrams of the given fatty acid ester (and/or fatty acid and/or triglyceride, respectively) per 100 milligrams of oil. This unit of measurement is used to describe the concentration of EPA in an EPA concentrate.

In some cases, it is useful to express the content of a given fatty acid(s) in a cell as its weight percent of the dry cell weight [“% DCW”]. Thus, for example, EPA % DCW would be determined according to the following formula: (EPA % TFAs)*(TFAs % DCW)]/100. The content of a given fatty acid(s) in a cell as its weight percent of the dry cell weight [“% DCW”] can be approximated, however, as: (EPA % TFAs)*(FAMEs % DCW)]/100.

The terms “lipid profile” and “lipid composition” are interchangeable and refer to the amount of individual fatty acids contained in a particular lipid fraction, such as in the total lipid or the oil, wherein the amount is expressed as a weight percent of TFAs. The sum of each individual fatty acid present in the mixture should be 100.

The term “oleaginous” refers to those organisms that tend to store their energy source in the form of lipid (Weete, In: Fungal Lipid Biochemistry, 2^{nd} Ed., Plenum, 1980). It is not uncommon for oleaginous microorganisms to accumulate in excess of about 25% of their dry cell weight as oil. Within oleaginous microorganisms the cellular oil or TAG content generally follows a sigmoid curve, wherein the concentration of lipid increases until it reaches a maximum at the late logarithmic or early stationary growth phase and then gradually decreases during the late stationary and death phases (Yongmanitchai and Ward, Appl. Environ. Microbiol. 57:419-25 (1991)).

The term “oleaginous yeast” refers to those microorganisms classified as yeasts that make oil. Examples of oleaginous yeast include, but are no means limited to, the following genera: Yarrowia, Candida, Rhodotorula, Rhodosporidium, Cryptococcus, Trichosporon and Lipomyces.

The term “substantially free of DHA” means comprising no more than about 0.05 weight percent of DHA. Thus, an EPA concentrate is substantially free of DHA when the concentration of DHA (in the form of free fatty acids, triacylglycerols, esters, and combinations thereof) is no more than about 0.05 wt % of DHA, measured as a wt % of the oil. Similarly, a microbial oil is substantially free of DHA (in the form of free fatty acids, triacylglycerols, esters, and combinations thereof) when the concentration of DHA is no more than about 0.05 wt % of DHA, measured as a wt % of TFAs.

The terms “substantially free of NDPA” and “substantially free of HPA” are comparable to the definition provided above for the term “substantially free of DHA”, although the fatty acid NDPA or HPA, respectively, is substituted for DHA.

The term “substantially free of environmental pollutants” means the oil or EPA concentrate, respectively, comprises either no environmental pollutants or at most only a trace of environmental pollutants, wherein these include compounds such as polychlorinated biphenyls [“PCBs”](CAS No. 1336-36-3), dioxins, brominated flame retardants and pesticides (e.g., toxaphenes and dichlorodiphenyltrichloroethane [“DDT”] and its metabolites).

The present invention concerns an EPA concentrate comprising at least 70 wt % of EPA, measured as a wt % of oil, and substantially free of DHA, said concentrate being obtained from a microbial oil comprising 30 to 70 wt % of EPA, measured as a wt % of TFAs, and substantially free of DHA, wherein said microbial oil is obtained from a microorganism that accumulates in excess of 25% of its dry cell weight as oil. The EPA concentrate is preferably substantially free of environmental pollutants and/or preferably substantially free from at least one fatty acid selected from the group consisting of NDPA and HPA.

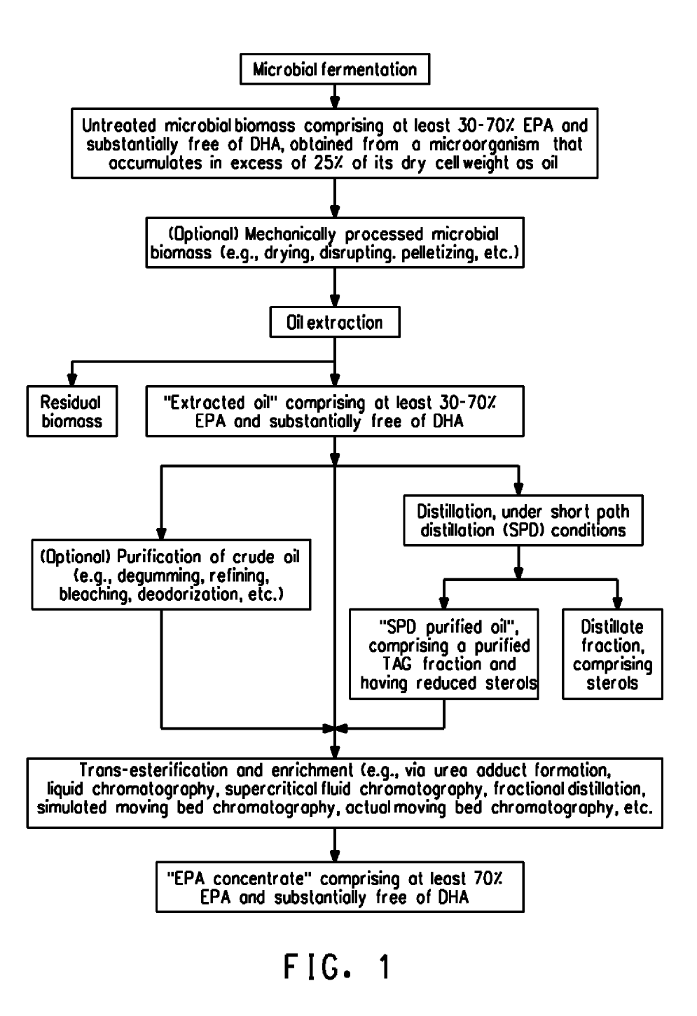

Although the present invention relates to the above, one will appreciate an overview of the related processes that may be useful to obtain the microbial oil itself (although this should not be construed as a limitation to the invention herein). As diagrammed in FIG. 1 in the form of a flowchart, most processes will begin with a microbial fermentation, wherein a particular microorganism is cultured under conditions that permit growth and production of PUFAs. At an appropriate time, the microbial cells are harvested from the fermentation vessel. This untreated microbial biomass, comprising at least 30-70 wt % of EPA and substantially free of DHA, may be subjected to various mechanical processing, such as drying, disrupting, pelletizing, etc. Oil extraction of the untreated microbial biomass is then performed, producing residual biomass (e.g., cell debris) and extracted oil. The extracted oil can then be directly transesterified and enriched to produce an EPA concentrate comprising at least 70 wt % EPA, measured as a wt % of oil, and substantially free of DHA; or, the extracted oil can first be purified and then subjected to transesterification and enrichment. For example, a purified oil can be produced by i) degumming, refining, bleaching, and/or deodorization, etc.; or, ii) distillation using short path distillation (SPD) conditions, thereby producing a purified TAG-fraction (i.e., the SPD-purified microbial oil) and a distillate fraction comprising sterols. Each of these aspects of FIG. 1 will be discussed in further detail below.

The microbial oil useful in the invention herein is typically derived from microbial biomass provided by microbial fermentation. A variety of oleaginous microbes (such as a fungi, algae, euglenoids, stramenopiles, yeast or any other single-cell organisms) can be grown in a microbial fermentation, to produce lipids containing at least 30 wt % of EPA, measured as a wt % of TFAs. Thus, any microorganism that accumulates in excess of 25% of its dry cell weight as oil, whether naturally occurring or recombinant, capable of producing at least 30 wt % of EPA, measured as a wt % of TFAs, may provide a suitable source of microbial oil for use in the enrichment processes described herein. Preferably, the microorganism will be capable of high level EPA production, wherein said production is preferably at least about 30-50 EPA % TFAs of the microbial host, more preferably at least about 50-60 EPA % TFAs, and most preferably at least about 60-70 EPA % TFAs.

On the other hand, oleaginous microorganisms capable of producing less than at least 30 wt % of EPA, measured as a wt % of TFAs, may also provide a suitable source of non-concentrated microbial oil that may be processed/concentrated to comprise at least 30 wt % of EPA, measured as a wt % of TFAs, for use in making the EPA concentrate of the invention.

Although the microorganism must necessarily comprise at least EPA, a variety of other polyunsaturated fatty acids may also be present in the organism, such as, e.g., linoleic acid, gamma-linolenic acid, eicosadienoic acid, dihomo-gamma-linolenic acid, arachidonic acid, docosatetraenoic acid, omega-6 docosapentaenoic acid, alpha-linolenic acid, stearidonic acid, eicosatrienoic acid, eicosatetraenoic acid, omega-3 docosapentaenoic acid, and mixtures thereof.

Although EPA is naturally produced in a variety of non-oleaginous and oleaginous microorganisms, including the heterotrophic diatoms Cyclotella sp. and Nitzschia sp. (U.S. Pat. No. 5,244,921), Pseudomonas, Alteromonas and Shewanella species (U.S. Pat. No. 5,246,841), filamentous fungi of the genus Pythium (U.S. Pat. No. 5,246,842), Mortierella elongata, M. exigua, and M. hygrophila (U.S. Pat. No. 5,401,646), and eustigmatophycean alga of the genus Nannochloropsis (Krienitz, L. and M. Wirth, Limnologica, 36:204-210 (2006)), microbial production of EPA using recombinant means is expected to have several advantages over production from natural microbial sources.

Recombinant microbes will have preferred characteristics for oil production, since the naturally occurring microbial fatty acid profile of the host can be altered by the introduction of new biosynthetic pathways in the host, overexpression of desirable pathways, and/or by the suppression of undesired pathways, thereby resulting in increased levels of production of desired PUFAs (or conjugated forms thereof) and decreased production of undesired PUFAs. Secondly, recombinant microbes can provide PUFAs in particular forms which may have specific uses. Additionally, microbial oil production can be manipulated by controlling culture conditions, notably by providing particular substrate sources for microbially expressed enzymes, or by addition of compounds/genetic engineering to suppress undesired biochemical pathways. Thus, for example, it is possible to modify the ratio of omega-3 to omega-6 fatty acids so produced, or engineer production of a specific PUFA (e.g., EPA) without significant accumulation of other PUFA downstream or upstream products (e.g., DHA). Highly controlled culture conditions also ensure that microbial oils obtained from these recombinant microbes are free of environmental pollutants.

Thus, for example, a microbe lacking the natural ability to make EPA can be engineered to express a PUFA biosynthetic pathway by introduction of appropriate PUFA biosynthetic pathway genes, such as delta-5 desaturases, delta-6 desaturases, delta-12 desaturases, delta-15 desaturases, delta-17 desaturases, delta-9 desaturases, delta-8 desaturases, delta-9 elongases, C_{14/16} elongases, C_{16/18} elongases and C_{18/20} elongases, although it is to be recognized that the specific enzymes (and genes encoding those enzymes) introduced are by no means limiting to the invention herein.

As an example, several types of yeast have been recombinantly engineered to produce EPA. See for example, work in the non-oleaginous yeast Saccharomyces cerevisiae (U.S. Pat. No. 7,736,884) and the oleaginous yeast, Yarrowia lipolytica (U.S. Pat. No. 7,238,482; U.S. Pat. No. 7,932,077; U.S. Pat. Appl. Pub. No. 2009-0093543-A1; U.S. Pat. Appl. Pub. No. 2010-0317072-A1). These examples should not be construed as a limitation herein.

In some embodiments, advantages are perceived if the microbial host cells are oleaginous. Oleaginous yeast are naturally capable of oil synthesis and accumulation, wherein the total oil content can comprise greater than about 25% of the cellular dry weight, more preferably greater than about 30% of the cellular dry weight, and most preferably greater than about 40% of the cellular dry weight. In alternate embodiments, a non-oleaginous yeast can be genetically modified to become oleaginous such that it can produce more than 25% oil of the cellular dry weight, e.g., yeast such as Saccharomyces cerevisiae (Int'l. Appl. Pub. No. WO 2006/102342).

Genera typically identified as oleaginous yeast include, but are not limited to: Yarrowia, Candida, Rhodotorula, Rhodosporidium, Cryptococcus, Trichosporon and Lipomyces. More specifically, illustrative oil-synthesizing yeasts include: Rhodosporidium toruloides, Lipomyces starkeyii, L. lipoferus, Candida revkaufi, C. pulcherrima, C. tropicalis, C. utilis, Trichosporon pullans, T. cutaneum, Rhodotorula glutinus, R. graminis, and Yarrowia lipolytica (formerly classified as Candida lipolytica).

As an example, in preferred embodiments herein, the source of the microbial oil comprising at least 30 wt % of EPA, measured as a wt % of TFAs, is from engineered strains of oleaginous yeast Yarrowia lipolytica. More preferred are microbial oils obtained from, for example, those strains described in U.S. Pat. Appl. Pub. No. 2009-0093543-A1 (some of which produce non-concentrated microbial oil comprising at least about 43.3 wt % EPA and substantially free of DHA) and in U.S. Pat. Appl. Pub. No. 2010-0317072-A1 (some of which produce non-concentrated microbial oil comprising at least 50 wt % EPA and substantially free of DHA). It is also contemplated herein that any of these recombinant Y. lipolytica strains could be subjected to further genetic engineering improvements (such as those described in Example 5 herein) and thus be a suitable source of microbial oil for the compositions and methods described herein. Thus, the preferred microbial oil is obtained from microbial biomass of recombinant Yarrowia cells, engineered for the production of EPA, wherein the microbial oil:
a) comprises 30 to 70 wt % EPA, measured as a wt % of TFAs, and is substantially free of DHA;
b) comprises from about 1 to about 25 wt % linoleic acid, measured as a wt % of TFAs;
c) has a ratio of at least 1.2 of EPA, measured as a wt % of TFAs, to linoleic acid, measured as a wt % of TFAs; and,
d) preferably is substantially free of NDPA and/or HPA.

More specifically, U.S. Pat. Appl. Pub. No. 2009-0093543-A1 describes high-level EPA production in optimized recombinant Yarrowia lipolytica strains. Strains are disclosed having the ability to produce microbial oils comprising at least about 43.3 EPA % TFAs, with less than about 23.6 LA % TFAs (an EPA:LA ratio of 1.83) and less than about 9.4 oleic acid % TFAs. The preferred strain was Y4305, which was capable of producing 33.2 EPA % TFAs, with an EPA:LA ratio of 1.25, mid-way through fermentation and whose maximum production was 55.6 EPA % TFAs, with an EPA:LA ratio of 3.03. Generally, the EPA-producing strains of U.S. Pat. Appl. Pub. No. 2009-0093543-A1 comprised the following genes of the omega-3/omega-6 fatty acid biosynthetic pathway: a) at least one gene encoding delta-9 elongase; b) at least one gene encoding delta-8 desaturase; c) at least one gene encoding delta-5 desaturase; d) at least one gene encoding delta-17 desaturase; e) at least one gene encoding delta-12 desaturase; f) at least one gene encoding C_{16/18} elongase; and, g) optionally, at least one gene encoding diacylglycerol cholinephosphotransferase [“CPT1”]. Since the pathway is genetically engineered into the host cell, there is no DHA concomitantly produced due to the lack of the appropriate enzymatic activities for elongation of EPA to DPA (catalyzed by a C_{20/22} elongase) and desaturation of DPA to DHA (catalyzed by a delta-4 desaturase). The disclosure also generally described microbial oils obtained from these engineered yeast strains and oil concentrates thereof.

A derivative of Yarrowia lipolytica strain Y4305 is described in U.S. patent application Ser. No. 12/854,449 (Attorney Docket No. “CL5143USNA”, filed Aug. 11, 2010 and hereby incorporated herein by reference), known as Y. lipolytica strain Y4305 F1B1. Upon growth in a two liter fermentation (parameters similar to those of U.S. Pat. Appl. Pub. No. 2009-009354-A1, Example 10), average EPA productivity [“EPA % DCW”] for strain Y4305 was 50-56, as compared to 50-52 for strain Y4305-F1B1. Average lipid content [“TFAs % DCW”] for strain Y4305 was 20-25, as compared to 28-32 for strain Y4305-F1B1. Thus, lipid content was increased 29-38% in strain Y4503-F1B1, with minimal impact upon EPA productivity.

U.S. Pat. Appl. Pub. No. 2010-0317072-A1 and U.S. Pat. Appl. Pub. No. 2010-0317735-A1 teach optimized strains of recombinant Y. lipolytica having the ability to produce further improved microbial oils relative to those strains described in U.S. Pat. Appl. Pub. No. 2009-0093543-A1, based on the EPA % TFAs and the ratio of EPA:LA. In addition to expressing genes of the omega-3/omega-6 fatty acid biosynthetic pathway as detailed in U.S. Pat. Appl. Pub. No. 2009-0093543-A1, these improved strains are distinguished by: a) comprising at least one multizyme, wherein said multizyme comprises a polypeptide having at least one fatty acid delta-9 elongase linked to at least one fatty acid delta-8 desaturase [a “DGLA synthase”]; b) optionally comprising at least one polynucleotide encoding an enzyme selected from the group consisting of a malonyl CoA synthetase or an acyl-CoA lysophospholipid acyltransferase [“LPLAT”]; c) comprising at least one peroxisome biogenesis factor protein whose expression has been down-regulated; d) producing at least about 50 EPA % TFAs; and, e) having a ratio of EPA:LA of at least about 3.1.

Specifically, in addition to possessing at least about 50 EPA % TFAs, the lipid profile within the improved optimized strains of Y. lipolytica of U.S. Pat. Appl. Pub. No. 2010-0317072-A1 and U.S. Pat. Appl. Pub. No. 2010-0317735-A1, or within extracted oil therefrom, will have a ratio of EPA % TFAs to LA % TFAs of at least about 3.1. Lipids produced by the improved optimized recombinant Y. lipolytica strains are also distinguished as having less than 0.05% GLA, NDPA, HPA or DHA and having a saturated fatty acid content of less than about 8%. This low percent of saturated fatty acids (i.e., 16:0 and 18:0) benefits both humans and animals.

More recently, U.S. patent application Ser. No. 13/218,708 (Attorney Docket Number CL5411USNA, filed on Aug. 26, 2011 and hereby incorporated herein by reference) describes further improved optimized recombinant microbial host cells having the ability to produce improved microbial oils relative to those strains described in U.S. Pat. Appl. Pub. No. 2009-0093543-A1 and U.S. Pat. Appl. Pub. No. 2010-0317072-A1, based on increased EPA productivity (i.e., measured as increased EPA % DCW). In addition to expressing genes of the omega-3/omega-6 fatty acid biosynthetic pathway, wherein said genes comprise at least one multizyme (wherein said multizyme comprises a polypeptide having at least one fatty acid delta-9 elongase linked to at least one fatty acid delta-8 desaturase [a “DGLA synthase”], as described in U.S. Pat. Appl. Pub. No. 2010-0317072-A1) and comprising at least one peroxisome biogenesis factor protein whose expression has been down-regulated (as described in U.S. Pat. Appl. Publications No. 2009-0117253-A1 and No. 2010-0317072-A1), the improved recombinant microbial host cells disclosed therein are further distinguished by:
1) comprising at least two polypeptides having at least lysophosphatidic acid acyltransferase [“LPAAT”] activity;
2) comprising at least one polypeptide having at least phospholipid:diacylglycerol acyltransferase [“PDAT”] activity;
3) optionally comprising at least one synthetic mutant delta-9 elongase polypeptide derived from Euglena gracilis; and,
4) producing a microbial oil comprising at least 25 wt % of EPA measured as a wt % of DCW.

One of skill in the art will appreciate that the methodology of the present invention is not limited to the Y. lipolytica strains described above, nor to the species (i.e., Y. lipolytica) or genus (i.e., Yarrowia) in which the invention has been demonstrated, as the means to introduce a PUFA biosynthetic pathway into an oleaginous yeast are well known. Instead, any oleaginous yeast or any other suitable oleaginous microbe such as fungi, algae, euglenoids, stramenopiles, or any other single-cell organisms capable of producing at least 30 wt % of EPA, measured as a wt % of TFAs and wherein the microbial oil obtained therefrom accumulates in excess of 25% of the microorganism's dry cell weight as oil, will be equally useful in the present methodologies.

To produce microbial oil comprising 30 to 70 wt % of EPA, measured as a wt % of TFAs, and substantially free of DHA, the oil-producing microbe will be grown under standard conditions well known by one skilled in the art of microbiology or fermentation science to optimize the production of EPA. With respect to genetically engineered microbes, the microbe will be grown under conditions that optimize expression of chimeric genes (e.g., encoding desaturases, elongases, DGLA synthases, CPT1 proteins, malonyl CoA synthetases, acyltransferases, etc.) and produce the greatest and the most economical yield of EPA. Typically, the microorganism is fed with a carbon and nitrogen source, along with a number of additional chemicals or substances that allow growth of the microorganism and/or production of EPA. The fermentation conditions will depend on the microorganism used, as described in the above citations, and may be optimized for a high content of the PUFA in the resulting biomass.

In general, media conditions may be optimized by modifying the type and amount of carbon source, the type and amount of nitrogen source, the carbon-to-nitrogen ratio, the amount of different mineral ions, the oxygen level, growth temperature, pH, length of the biomass production phase, length of the oil accumulation phase and the time and method of cell harvest.

More specifically, fermentation media should contain a suitable carbon source, such as are taught in U.S. Pat. No. 7,238,482 and U.S. Pat. Appl. Pub. No. 2011-0059204-A1. Although it is contemplated that the source of carbon utilized for growth of an engineered EPA-producing microbe may encompass a wide variety of carbon-containing sources, preferred carbon sources are sugars, glycerol and/or fatty acids. Most preferred are glucose, sucrose, invert sucrose, fructose and/or fatty acids containing between 10-22 carbons. For example, the fermentable carbon source can be selected from the group consisting of invert sucrose (i.e., a mixture comprising equal parts of fructose and glucose resulting from the hydrolysis of sucrose), glucose, fructose and combinations of these, provided that glucose is used in combination with invert sucrose and/or fructose.

Nitrogen may be supplied from an inorganic (e.g., (NH₄)₂SO₄) or organic (e.g., urea or glutamate) source. In addition to appropriate carbon and nitrogen sources, the fermentation media must also contain suitable minerals, salts, cofactors, buffers, vitamins and other components known to those skilled in the art suitable for the growth of the oleaginous yeast and promotion of the enzymatic pathways necessary for EPA production.

Particular attention is given to several metal ions (e.g., Fe⁺², Cu⁺², Mn⁺², Co⁺², Zn⁺² and Mg⁺²) that promote synthesis of lipids and PUFAs (Nakahara, T. et al., Ind. Appl. Single Cell Oils, D. J. Kyle and R. Colin, eds. pp 61-97 (1992)).

Preferred growth media are common commercially prepared media, such as Yeast Nitrogen Base (DIFCO Laboratories, Detroit, Mich.). Other defined or synthetic growth media may also be used and the appropriate medium for growth of Yarrowia lipolytica will be known by one skilled in the art of microbiology or fermentation science. A suitable pH range for the fermentation is typically between about pH 4.0 to pH 8.0, wherein pH 5.5 to pH 7.5 is preferred as the range for the initial growth conditions. The fermentation may be conducted under aerobic or anaerobic conditions.

Typically, accumulation of high levels of PUFAs in oleaginous yeast cells requires a two-stage process, since the metabolic state must be “balanced” between growth and synthesis/storage of fats. Thus, most preferably, a two-stage fermentation process is necessary for the production of EPA in Y. lipolytica. This approach is described in U.S. Pat. No. 7,238,482, as are various suitable fermentation process designs (i.e., batch, fed-batch and continuous) and considerations during growth.

When the desired amount of EPA has been produced by the microorganism, the fermentation medium may be treated to obtain microbial biomass comprising the PUFA. For example, the fermentation medium may be filtered or otherwise treated to remove at least part of the aqueous component. The fermentation medium and/or the microbial biomass may be further processed; for example, the microbial biomass may be pasteurized or treated via other means to reduce the activity of endogenous microbial enzymes that can harm the microbial oil and/or PUFA products. The microbial biomass may be mechanically processed, for example by drying the biomass, disrupting the biomass (e.g., via cellular lysing), pelletizing the biomass, or a combination of these. The microbial biomass may be dried, e.g., to a desired water content, granulated or pelletized for ease of handling, and/or mechanically disrupted e.g., via physical means such as bead beaters, screw extrusion, etc. to provide greater accessibility to the cell contents. The microbial biomass will be referred to as untreated microbial biomass, even after any of these mechanical processing steps, since oil extraction has not yet occurred.

One preferred method for mechanical processing of microbial biomass is described in U.S. Provisional Appl. No. 61/441,836 (Attorney Docket Number CL5053USPRV, filed on Feb. 11, 2011) and U.S. patent application Ser. No. ______ (Attorney Docket Number CL5053USNA (co-filed herewith) (each incorporated herein by reference). Specifically, the method involves twin-screw extrusion of dried yeast with a grinding agent (e.g., silica, silicate) capable of absorbing oil to provide a disrupted biomass mix, followed by blending a binding agent (e.g., sucrose, lactose, glucose, soluble starch) with said disrupted biomass mix to provide a fixable mix capable of forming a solid pellet, and subsequent forming of solid pellets (e.g., of ˜1 mm diameter X 6-10 mm length) from the fixable mix (“pelletization”).

Following optional mechanical processing, the microbial oil is generally (although not necessarily) separated from other cellular materials that might be present in the microorganism which produced the oil via oil extraction.

Oil extraction can occur via treatment with various organic solvents (e.g., hexane, iso-hexane), enzymatic extraction, osmotic shock, ultrasonic extraction, supercritical fluid extraction (e.g., CO₂ extraction), saponification and combinations of these methods. These processes will result in residual biomass (i.e., cell debris, etc.) and extracted oil preferably comprising 30 to 70 wt % of EPA, measured as a wt % of TFAs, and substantially free of DHA.

When using supercritical fluid extraction, any suitable supercritical fluid or liquid solvent may be used to separate the EPA-containing oil from the biomass (e.g., CO₂, tetrafluoromethane, ethane, ethylene, propane, propylene, butane, isobutane, isobutene, pentane, hexane, cyclohexane, benzene, toluene, xylenes, and mixtures thereof, provided that the supercritical fluid is inert to all reagents and products); more preferred solvents include CO₂ or a C₃-C₆ alkane (e.g., pentane, butane, and propane). Most preferred solvents are supercritical fluid solvents comprising CO₂. The extraction does not concentrate the fatty acid composition and the extracted oil which is recovered is thus a non-concentrated microbial oil.

In a preferred embodiment, super-critical carbon dioxide extraction is performed, as disclosed in U.S. Pat. Pub. No. 2011-0263709-A1 (hereby incorporated herein by reference). This particular methodology subjects untreated disrupted microbial biomass to oil extraction to remove residual biomass comprising phospholipids, and then fractionates the resulting extract at least once to obtain an extracted oil having a refined lipid composition comprising at least one PUFA, wherein the refined lipid composition is enriched in TAGs relative to the oil composition of the untreated disrupted microbial biomass.

In some embodiments, the extracted oil comprising 30 to 70 wt % of EPA, measured as a wt % of TFAs, and substantially free of DHA, may optionally undergo further purification steps. For example, one of skill in the art will be familiar with procedures of degumming (e.g., to remove phospholipids, trace metals and free fatty acids), refining, bleaching (e.g., to adsorb color compounds and minor oxidation products), and/or deodorization (e.g., to remove volatile, odorous and/or additional color compounds). As none of these methods substantially enrich the EPA concentration within the microbial oil, the product of these processes is still typically considered a non-concentrated microbial oil, albeit in a purified form. The EPA and other PUFAs within this oil primarily remain in their natural triglyceride form.

Alternatively, it may be desirable to distill the extracted oil comprising 30 to 70 wt % of EPA, measured as a wt % of TFAs, and substantially free of DHA, to remove moisture and e.g., sterols. Sterols, which function in the membrane permeability of cells, have been isolated from all major groups of living organisms, although there is diversity in the predominant sterol isolated. The predominant sterol in higher animals is cholesterol, while R-sitosterol is commonly the predominant sterol in higher plants (although it is frequently accompanied by campesterol and stigmasterol). Generalization concerning the predominant sterol(s) found in microbes is more difficult, as the composition depends on the particular microbial species. For example, the oleaginous yeast Yarrowia lipolytica predominantly comprises ergosterol, fungus of the genus Morteriella predominantly comprise cholesterol and desmosterol, and stramenopiles of the genus Schizochytrium predominantly comprise brassicasterol and stigmasterol. Sterols (e.g., ergosterol) have been observed to phase separate from TAGs, especially at low storage temperatures, thereby resulting in undesirable cloudiness in the microbial oil.

U.S. Provisional Appl. No. 61/441,842 (Attorney Docket Number CL5077USPRV, filed on Feb. 11, 2011) and U.S. patent application Ser. No. ______ (Attorney Docket Number CL5077USNA (co-filed herewith) (each incorporated herein by reference) describe a process to reduce sterol content in a sterol-containing extracted oil, the process including at least one pass of the sterol-containing microbial oil through a short path distillation (SPD) still.

Commercial SPD stills are well known in the art of chemical engineering. Suitable stills are available, for example, from Pope Scientific (Saukville, Wis.). The SPD still includes an evaporator and an internal condenser. A typical distillation is controlled by the temperature of the evaporator, the temperature of the condenser, the feed-rate of the oil into the still and the vacuum level of the still.

As one of skill in the art will appreciate, the number of passes through a SPD still will depend on the level of moisture in the sterol-containing microbial oil. If the moisture content is low, a single pass through the SPD still may be sufficient. Preferably, however, the distillation is a multi-pass process including two or more consecutive passes of the sterol-containing extracted oil through a SPD still. A first pass is typically performed under about 1 to 50 torr pressure, and preferably about 5 to 30 torr, with relatively low surface temperature of the evaporator, for instance, about 100 to 150° C. This results in a dewatered oil, as residual water and low molecular weight organic materials are distilled. The dewatered oil is then passed through the still at higher temperature of the evaporator and lower pressures to provide a distillate fraction enriched in the sterol and a TAG-containing fraction having a reduced amount of the sterol, as compared to the oil not subject to SPD. Additional passes of the TAG-containing fraction may be made through the still to remove further sterol. Preferably, sufficient passes are performed such that the reduction in the amount of the sterol fraction is at least about 40%-70%, preferably at least about 70%-80%, and more preferably greater than about 80%, when compared to the sterol fraction in the sterol-containing microbial oil.

More preferably: i) the SPD conditions comprise at least one pass of the sterol-containing microbial oil at a vacuum level of not more than 30 mTorr, and preferably not more than 5 mTorr; ii) the SPD conditions comprise at least one pass at about 220 to 300° C., and preferably at about 240 to 280° C.; and, iii) the SPD conditions have an evaporator temperature of not more than 300° C., and more preferably, not more than 280° C.

The SPD process results in a TAG-containing fraction (i.e., SPD-purified oil) having a reduced sterol fraction that has improved clarity when compared to the sterol-containing microbial oil composition that has not been subjected to SPD. Improved clarity refers to a lack of cloudiness or opaqueness in the oil. Sterol-containing microbial oil becomes cloudy upon storing at temperatures below about 10° C., due to the elevated levels of sterol in the oil. The distillation process acts to remove substantial portions of the sterol fraction, such that the resulting TAG-containing fraction has a reduced amount of sterol present, and thus, remains clear, or substantially clear upon storage at about 10° C. A test method that may be used to evaluate the clarity of the oil is the American Oil Chemists' Society (AOCS) Official Method Cc 11-53 entitled “Cold Test” (Official Methods and Recommended Practices of the AOCS, 6^{th} ed., Urbana, Ill., AOCS Press, 2009, incorporated herein by reference).

Surprisingly, the removal of sterol in the distillation process can be accomplished without significant degradation of the oil, based on evaluation of the PUFA content before and after the process.

Recovering the TAG-containing fraction, which is a purified microbial oil comprising 30 to 70 wt % of EPA, measured as a wt % of TFAs, and substantially free of DHA, may be accomplished by diverting the fraction, after completion of a pass through the evaporator, to a suitable container.

The fatty acids in microbial oil (i.e., extracted oil or purified oil) are typically in a biological form such as a triglyceride or phospholipid. Because it is difficult to enrich the fatty acid profile of these forms, the individual fatty acids of the microbial oil will usually be liberated by transesterification using techniques well known to those skilled in the art. Since the fatty acid ester mixture has substantially the same fatty acid profile as the microbial oil prior to transesterification, the product of the transesterification process is still typically considered a non-concentrated microbial oil (i.e., in ester form).

Enrichment of the microbial oil comprising 30 to 70 wt % of EPA, measured as a wt % of TFAs, and substantially free of DHA (wherein the microbial oil is obtained from a microorganism that accumulates in excess of 25% of its dry cell weight as oil) results in an oil concentrate which comprises at least 70 wt % of EPA, measured as a wt % of oil, and is substantially free of DHA (i.e., an “EPA concentrate”). Specifically, the ethyl or other esters of the microbial oil can be enriched in EPA and separated by methods commonly used in the art, such as: fractional distillation, urea adduct formation, short path distillation, supercritical fluid fractionation with counter current column, supercritical fluid chromatography, liquid chromatography, enzymatic separation and treatment with silver salt, simulated moving bed chromatography, actual moving bed chromatography and combinations thereof.

Thus, also provided herein is a method for making an EPA concentrate comprising at least 70 wt % EPA, measured as a wt % of oil and substantially free of DHA, said method comprising:
a) transesterifying a microbial oil comprising 30 to 70 wt % EPA, measured as a wt % of TFAs, and substantially free of DHA, wherein said microbial oil is obtained from a microorganism that accumulates in excess of 25% of its dry cell weight as oil; and,
b) enriching the transesterified oil of step (a) to obtain an EPA concentrate comprising at least 70 wt % EPA, measured as a wt % of oil, and substantially free of DHA.

For example, a non-concentrated purified microbial oil comprising 58.2% EPA, measured as a wt % of TFAs, and substantially free of DHA from Yarrowia lipolytica is provided in the Examples herein. This non-concentrated microbial oil is enriched in Example 2 via a urea adduct formation method, such that the resulting EPA-EE concentrate comprises 76.5% EPA-EE, measured as a wt % of oil, and is substantially free of DHA. Similarly, Example 3 demonstrates enrichment of the same non-concentrated microbial oil via liquid chromatography, wherein the resulting EPA-EE concentrate comprises 82.8% or 95.4% EPA-EE, measured as a wt % of oil, and is substantially free of DHA. Example 4 demonstrates enrichment of the same non-concentrated microbial oil via supercritical fluid chromatography, resulting in an EPA concentrate comprising 85% or 89.8% EPA-EE, measured as a wt % of oil, that is substantially free of DHA.

An alternate non-concentrated SPD-purified microbial oil comprising 56.1% EPA, measured as a wt % of TFAs, and substantially free of DHA from Yarrowia lipolytica is provided in Example 5. Enrichment of this microbial oil in Example 6 occurs via fractional distillation, thereby producing an EPA concentrate that comprises 73% EPA-EE, measured as a wt % of oil, and is substantially free of DHA. Fractional distillation advantageously removes many of the lower molecular weight ethyl esters present in the oil (i.e., predominantly C18s in the microbial oil of Example 6, but not limited thereto).

An alternate non-concentrated SPD-purified microbial oil comprising 54.7% EPA, measured as a wt % of TFAs, and substantially free of DHA, NDPA and HPA from Yarrowia lipolytica is provided in Example 8. Enrichment of this microbial oil occurs via fractional distillation and liquid chromatography, thereby producing an EPA concentrate that comprises 97.4% EPA-EE, measured as a wt % of oil, and is substantially free of DHA, NDPA and HPA. One of skill in the art should appreciate that other combinations of enrichment processes (e.g., fractional distillation, urea adduct formation, short path distillation, supercritical fluid fractionation with counter current column, supercritical fluid chromatography, liquid chromatography, enzymatic separation and treatment with silver salt, simulated moving bed chromatography, actual moving bed chromatography) could be utilized to produce an EPA concentrate of the present invention.

For example, it may be particularly advantageous to make an EPA concentrate comprising at least 70 wt % of EPA, measured as a wt % of oil, and substantially free of DHA, said method comprising: (a) a transesterification reaction of a microbial oil comprising 30 to 70 wt % of EPA, measured as a wt % of TFAs; (b) a first enrichment process comprising fractional distillation for removal of many of the lower molecular weight ethyl esters, i.e., comprising C14, C16 and C18 fatty acids; and, (c) at least one additional enrichment process selected from the group consisting of: urea adduct formation, liquid chromatography, supercritical fluid chromatography, simulated moving bed chromatography, actual moving bed chromatography and combinations thereof. Lower concentrations of C14, C16 and C18 fatty acids in the microbial oil sample, as a result of fractional distillation, may facilitate subsequent enrichment processes.

As will be recognized by one of skill in the art, any of the EPA concentrates described above, in ethyl ester form, can readily be converted, if desired, to other forms such as, for example, a methyl ester, an acid or a triacylglyceride, or any other suitable form or a combination thereof. Means for chemical conversion of PUFAs from one derivative to another is well known. For example, triglycerides can be converted to sodium salts of the cleaved acids by saponification and further to free fatty acids by acidification, and ethyl esters can be re-esterified to triglycerides via glycerolysis. Thus, while it is expected that the EPA concentrate will initially be in the form of an ethyl ester, this is by no means intended as a limitation. The at least 70 wt % EPA, measured as a wt % of oil, within an EPA concentrate will therefore refer to EPA in the form of free fatty acids, triacylglycerols, esters, and combinations thereof, wherein the esters are most preferably in the form of ethyl esters.

One of ordinary skill in the art will appreciate that processing conditions can be optimized to result in any preferred level of EPA enrichment of the microbial oil, such that the EPA concentrate has at least 70 wt % EPA, measured as a wt % of oil (although increased EPA purity is often inversely related to EPA yield). Thus, those skilled in the art will appreciate that the wt % of EPA can be any integer percentage (or fraction thereof) from 70% up to and including 100%, i.e., specifically, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% EPA, measured as a wt % of oil.

More specifically, in one embodiment of the present invention, there is provided an EPA concentrate comprising at least 80 wt % of EPA, measured as a wt % of oil, and substantially free of DHA. In another embodiment, there is provided an EPA concentrate comprising at least 90 wt % of EPA, measured as a wt % of oil, and substantially free of DHA. And, in yet another embodiment, there is provided an EPA concentrate comprising at least 95 wt % of EPA, measured as a wt % of oil, and substantially free of DHA.

In preferred embodiments, the EPA concentrates described above, comprising at least 70 wt % EPA, measured as a wt % of oil, and substantially free of DHA can be further characterized as substantially free of NDPA and substantially free of HPA.

Although not limited to any particular application, the EPA concentrate of the present invention is particularly well suited for use as a pharmaceutical. As is well known to one of skill in the art, EPA may be administered in a capsule, a tablet, a granule, a powder that can be dispersed in a beverage, or another solid oral dosage form, a liquid (e.g., syrup), a soft gel capsule, a coated soft gel capsule or other convenient dosage form such as oral liquid in a capsule. Capsules may be hard-shelled or soft-shelled and may be of a gelatin or vegetarian source. EPA may also be contained in a liquid suitable for injection or infusion.

Additionally, EPA may also be administered with a combination of one or more non-active pharmaceutical ingredients (also known generally herein as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use.

Excipients may include, but are not limited to, surfactants, such as propylene glycol monocaprylate, mixtures of glycerol and polyethylene glycol esters of long fatty acids, polyethoxylated castor oils, glycerol esters, oleoyl macrogol glycerides, propylene glycol monolaurate, propylene glycol dicaprylate/dicaprate, polyethylene-polypropylene glycol copolymer and polyoxyethylene sorbitan monooleate, cosolvents such as ethanol, glycerol, polyethylene glycol, and propylene glycol, and oils such as coconut, olive or safflower oils. The use of surfactants, cosolvents, oils or combinations thereof is generally known in the pharmaceutical arts, and as would be understood to one skilled in the art, any suitable surfactant may be used in conjunction with the present invention and embodiments thereof.

The dose concentration, dose schedule and period of administration of the composition should be sufficient for the expression of the intended action, and may be adequately adjusted depending on, for example, the dosage form, administration route, severity of the symptom(s), body weight, age and the like. When orally administered, the composition may be administered in three divided doses per day, although the composition may alternatively be administered in a single dose or in several divided doses.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### Example 1

### Preparation of a Microbial Oil Comprising 58.2% EPA of Total Fatty Acids [“TFAs”]

The present Example describes the isolation of a microbial oil obtained from microbial biomass of recombinant Yarrowia lipolytica cells, engineered for the production of EPA. This microbial oil was then enriched by various means, as described below in Examples 2-4.

Specifically, Y. lipolytica strain Y8672 was recombinantly engineered to enable production of about 61.8 EPA % TFAs and cultured using a 2-stage fed-batch process. Microbial oil was then isolated from the resulting microbial biomass via an iso-hexane solvent and purified, yielding a non-concentrated, triglyceride-rich purified oil comprising 58.2 EPA % TFAs.

Genotype of Yarrowia lipolytica Strain Y8672

The generation of strain Y8672 is described in U.S. Pat. Appl. Pub. No. 2010-0317072-A1. Strain Y8672, derived from Y. lipolytica ATCC #20362, was capable of producing about 61.8% EPA relative to the total lipids via expression of a delta-9 elongase/delta-8 desaturase pathway.

The final genotype of strain Y8672 with respect to wild type Y. lipolytica ATCC #20362 was Ura+, Pex3-, unknown 1-, unknown 2-, unknown 3-, unknown 4-, unknown 5-, unknown 6-, unknown 7-, unknown 8-, Leu+, Lys+, YAT1::ME3S::Pex16, GPD::ME3S::Pex20, GPD::FmD12::Pex20, YAT1::FmD12::Oct, EXP1::FmD12S::ACO, GPAT::EgD9e::Lip2, FBAINm::EgD9eS::Lip2, EXP1::EgD9eS::Lip1, YAT1::EgD9eS::Lip2, FBAINm::EgD8M::Pex20, FBAIN::EgD8M::Lip1, EXP1::EgD8M::Pex16, GPD::EaD8S::Pex16 (2 copies), YAT1::E389D9eS/EgD8M::Lip1, YAT1::EgD9eS/EgD8M::Aco, FBAIN::EgD5SM::Pex20, YAT1::EgD5SM::Aco, GPM::EgD5SM::Oct, EXP1::EgD5M::Pex16, EXP1::EgD5SM::Lip1, YAT1::EaD5SM::Oct, YAT1::PaD17S::Lip1, EXP1::PaD17::Pex16, FBAINm::PaD17::Aco, GPD::YICPT1::Aco, and YAT1::MCS::Lip1.

The structure of the above expression cassettes are represented by a simple notation system of “X::Y::Z”, wherein X describes the promoter fragment, Y describes the gene fragment, and Z describes the terminator fragment, which are all operably linked to one another. Abbreviations are as follows: FmD12 is a Fusarium moniliforme delta-12 desaturase gene [U.S. Pat. No. 7,504,259]; FmD12S is a codon-optimized delta-12 desaturase gene, derived from F. moniliforme [U.S. Pat. No. 7,504,259]; ME3S is a codon-optimized C_{16/18} elongase gene, derived from Mortierella alpina [U.S. Pat. No. 7,470,532]; EgD9e is a Euglena gracilis delta-9 elongase gene [U.S. Pat. No. 7,645,604]; EgD9eS is a codon-optimized delta-9 elongase gene, derived from E. gracilis [U.S. Pat. No. 7,645,604]; EgD8M is a synthetic mutant delta-8 desaturase gene [U.S. Pat. No. 7,709,239], derived from E. gracilis [U.S. Pat. No. 7,256,033]; EaD8S is a codon-optimized delta-8 desaturase gene, derived from Euglena anabaena [U.S. Pat. No. 7,790,156]; E389D9eS/EgD8M is a DGLA synthase created by linking a codon-optimized delta-9 elongase gene (“E389D9eS”), derived from Eutreptiella sp. CCMP389 delta-9 elongase (U.S. Pat. No. 7,645,604) to the delta-8 desaturase “EgD8M” (supra) [U.S. Pat. Appl. Pub. No. 2008-0254191-A1]; EgD9ES/EgD8M is a DGLA synthase created by linking the delta-9 elongase “EgD9eS” (supra) to the delta-8 desaturase “EgD8M” (supra) [U.S. Pat. Appl. Pub. No. 2008-0254191-A1]; EgD5M and EgD5SM are synthetic mutant delta-5 desaturase genes [U.S. Pat. Appl. Pub. No. 2010-0075386-A1], derived from Euglena gracilis [U.S. Pat. No. 7,678,560]; EaD5SM is a synthetic mutant delta-5 desaturase gene [U.S. Pat. Appl. Pub. No. 2010-0075386-A1], derived from Euglena anabaena [U.S. Pat. No. 7,943,365]; PaD17 is a Pythium aphanidermatum delta-17 desaturase gene [U.S. Pat. No. 7,556,949]; PaD17S is a codon-optimized delta-17 desaturase gene, derived from P. aphanidermatum [U.S. Pat. No. 7,556,949]; YICPT1 is a Yarrowia lipolytica diacylglycerol cholinephosphotransferase gene [U.S. Pat. No. 7,932,077]; and, MCS is a codon-optimized malonyl-CoA synthetase gene, derived from Rhizobium leguminosarum bv. viciae 3841 [U.S. Pat. Appl. Pub. No. 2010-0159558-A1].

For a detailed analysis of the total lipid content and composition in strain Y8672, a flask assay was conducted wherein cells were grown in 2 stages for a total of 7 days. Based on analyses, strain Y8672 produced 3.3 g/L dry cell weight [“DCW”], total lipid content of the cells was 26.5 [“TFAs % DCW”], the EPA content as a percent of the dry cell weight [“EPA % DCW”] was 16.4, and the lipid profile was as follows, wherein the concentration of each fatty acid is as a weight percent of TFAs [“% TFAs”]: 16:0 (palmitate)-2.3, 16:1 (palmitoleic acid)—0.4, 18:0 (stearic acid)—2.0, 18:1 (oleic acid)—4.0, 18:2 (LA)—16.1, ALA—1.4, EDA—1.8, DGLA—1.6, ARA—0.7, ETrA—0.4, ETA—1.1, EPA—61.8, other—6.4.

Fermentation and Extraction of Microbial Oil From Y. lipolytica Strain Y8672 Biomass

Inocula were prepared from frozen cultures of Y. lipolytica strain Y8672 in a shake flask. After an incubation period, the culture was used to inoculate a seed fermentor. When the seed culture reached an appropriate target cell density, it was then used to inoculate a larger fermentor. The fermentation was a 2-stage fed-batch process. In the first stage, the yeast were cultured under conditions that promoted rapid growth to a high cell density; the culture medium comprised glucose, various nitrogen sources, trace metals and vitamins. In the second stage, the yeast were starved for nitrogen and continuously fed glucose to promote lipid and PUFA accumulation. Process variables including temperature (controlled between 30-32° C.), pH (controlled between 5-7), dissolved oxygen concentration and glucose concentration were monitored and controlled per standard operating conditions to ensure consistent process performance and final PUFA oil quality.

One of skill in the art of fermentation will know that variability will occur in the oil profile of a specific Yarrowia strain, depending on the fermentation run itself, media conditions, process parameters, scale-up, etc., as well as the particular time-point in which the culture is sampled (see, e.g., U.S. Pat. Appl. Pub. No. 2009-0093543-A1).

After fermentation, the yeast biomass was dewatered and washed to remove salts and residual medium, and to minimize lipase activity. Drum drying followed to reduce the moisture to less than 5% to ensure oil stability during short term storage and transportation of the untreated microbial biomass.

The microbial biomass was then subjected to mechanical disruption with iso-hexane solvent to extract the EPA-rich microbial oil from the biomass. The residual biomass (i.e., cell debris) was removed and the solvent was evaporated to yield an extracted oil. The extracted oil was degummed using phosphoric acid and refined with 20° Baume caustic to remove phospholipids, trace metals and free fatty acids. Bleaching with silica and clay was used to adsorb color compounds and minor oxidation products. The last deodorization step stripped out volatile, odorous and additional color compounds to yield a non-concentrated purified microbial oil comprising PUFAs in their natural triglyceride form.

Characterization of Microbial Oil from Y. lipolytica Strain Y8672

The fatty acid composition of the non-concentrated purified oil was analyzed using the following gas chromatography [“GC”]method. Specifically, the triglycerides were converted to fatty acid methyl esters [“FAMEs”] by transesterification using sodium methoxide in methanol. The resulting FAMEs were analyzed using an Agilent 7890 GC fitted with a 30-m×0.25 mm (i.d.) OMEGAWAX (Supelco) column after dilution in toluene/hexane (2:3). The oven temperature was increased from 160° C. to 200° C. at 5° C./min, and then 200° C. to 250° C. (hold for 10 min) at 10° C./min.

FAME peaks recorded via GC analysis were identified by their retention times, when compared to that of known methyl esters [“MEs”], and quantitated by comparing the FAME peak areas with that of the internal standard (C15:0 triglyceride, taken through the transesterification procedure with the sample) of known amount. Thus, the approximate amount (mg) of any fatty acid FAME [“mg FAME”] is calculated according to the formula: (area of the FAME peak for the specified fatty acid/area of the 15:0 FAME peak)*(mg of the internal standard C15:0 FAME). The FAME result can then be corrected to mg of the corresponding fatty acid by dividing by the appropriate molecular weight conversion factor of 1.042-1.052.

The lipid profile, summarizing the amount of each individual fatty acid as a weight percent of TFAs, was determined by dividing the individual FAME peak area by the sum of all FAME peak areas and multiplying by 100.

The results obtained from the GC analyses on the non-concentrated Y8672 purified oil are shown below in Table 3. The purified oil contained 58.2 EPA % TFAs and DHA was non-detectable (i.e. <0.05%).

TABLE 3
Fatty Acid Composition Of Non-Concentrated Y8672 Purified Oil

| Fatty acid | Weight Percent Of Total Fatty Acids |
|---|---|
| C18:2 (omega-6) | 16.6 |
| C20:5 EPA | 58.2 |
| C22:6 DHA | non-detectable (<0.05%) |
| Other components | 25.2 |

### Example 2

### Enrichment of Microbial Oil Via Urea Adduct Formation

This example demonstrates that an EPA concentrate comprising up to 78% EPA ethyl esters, measured as a weight percent of oil, and substantially free of DHA could be obtained upon enrichment of the non-concentrated purified oil from Example 1 via urea adduct formation.

KOH (20 g) was first dissolved in 320 g of absolute ethanol. The solution was then mixed with 1 kg of the non-concentrated purified oil from Example 1 and heated to approximately 60° C. for 4 hrs. The reaction mixture was left undisturbed in a Sep funnel overnight for complete phase separation.

After removing the bottom glycerol fraction, a small amount of silica was added to the upper ethyl ester fraction to remove excess soap. The ethanol was rotovapped off at about 90° C. under vacuum, which yielded clear, but light-brown, ethyl esters.

The ethyl esters (20 g) were mixed with 40 g of urea and 100 g of ethanol (90% aqueous) at approximately 65° C. The mixture was maintained at this temperature until it turned into a clear solution. The mixture was then cooled to and held at room temperature for approximately 20 hrs for urea crystals and adducts to form. The solids were then removed through filtration and the liquid fraction was rotovapped to remove ethanol. The recovered ethyl ester fraction was washed with a first and then a second wash of 200 mL of warm water. The pH of the solution was adjusted to 3-4 first before decanting off the aqueous fraction. The ethyl ester fraction was then dried to remove residual water.

To determine the fatty acid ethyl ester [“FAEE”] concentrations in the ethyl ester fraction, the FAEEs were analyzed directly after dilution in toluene/hexane (2:3), using the same GC conditions and calculations as previously described in Example 1 to determine FAME concentrations. The only modifications in methodology were: i) C23:0 EE was used as the internal standard instead of C15:0; and, ii) the molecular weight conversion factor of 1.042-1.052 was not required.

EPA ethyl ester [“EPA-EE”], however, was subjected to a slightly modified procedure from that above. Specifically, a reference EPA-EE standard of known concentration and purity was prepared to contain approximately the same amount of EPA-EE expected in the analytical samples, as well as the same amount of C23:0 EE internal standard. The exact amount of EPA-EE (mg) in a sample is calculated according to the formula: (area of EPA-EE peak/area of the C23:0 EE peak)×(area of the C23:0 EE peak in the calibration standard/area of the EPA-EE peak in the calibration standard)×(mg EPA-EE in the calibration standard). All internal and reference standards were obtained from Nu-Chek Prep, Inc.

In this way, the FAEE concentrations were determined in the enriched oil fraction, i.e., the EPA concentrate. Specifically, enrichment of the non-concentrated purified oil via urea adduct formation yielded an EPA concentrate with 77% EPA ethyl ester, measured as a weight percent of oil, and substantially free of DHA, as shown in Table 4.

TABLE 4
EPA Ethyl Ester Concentrate With Urea Adduct Method

| Fatty acid ethyl esters | Weight Percent Of Oil |
|---|---|
| C18:2 (omega-6) | 3.9 |
| C20:5 EPA | 76.5 |
| C22:6 DHA | non-detectable (<0.05%) |
| Other components | 19.6 |

One of ordinary skill in the art will appreciate that the EPA concentrate, comprising 77% EPA ethyl ester, measured as a weight percent of oil, and substantially free of DHA, could readily be converted to yield an EPA concentrate in an alternate form (i.e., the EPA ethyl ester could be converted to free fatty acids, triacylglycerols, methyl esters, and combinations thereof), using means well known to those of skill in the art. Thus, for example, the 77% EPA ethyl ester could be re-esterified to triglycerides via glycerolysis, to result in an EPA concentrate, in triglyceride form, comprising at least 70 wt % of EPA, measured as a wt % of oil, and substantially free of DHA.

### Example 3

### Enrichment of Microbial Oil Via Liquid Chromatography

This example demonstrates that an EPA concentrate comprising up to 95.4% EPA ethyl ester, measured as a weight percent of oil, and substantially free of DHA could be obtained upon enrichment of the non-concentrated purified oil from Example 1 using a liquid chromatography method.

The non-concentrated purified oil from Example 1 was transesterified to ethyl esters using a similar method as described in Example 2 but with some minor modifications (i.e., use of sodium ethoxide as a base catalyst instead of potassium hydroxide).

The ethyl esters were then enriched by Equateq (Isle of Lewis, Scotland) using their liquid chromatographic purification technology. Various degrees of enrichment were achieved (e.g., see exemplary data for Sample #1 and Sample #2, infra). Thus, enrichment of the non-concentrated purified oil via liquid chromatography yielded an EPA concentrate with up to 95.4% EPA ethyl ester, measured as a weight percent of oil, and substantially free of DHA, as shown in Table 5.

TABLE 5
EPA Ethyl Ester Concentrate With A Liquid Chromatography
Enrichment Method

| Fatty | Weight Percent Of Oil | |
|---|---|---|
| acid ethyl esters | Sample #1 | Sample #2 |
| C18:2 (omega-6) | 5.7 | ND |
| C20:5 EPA | 82.8 | 95.4 |
| C22:6 DHA | non-detectable (<0.05%) | non-detectable (<0.05%) |
| Other components | 11.5 | 4.6 |

One of skill in the art will appreciate that the EPA concentrate, comprising either 82.8% EPA ethyl ester or 95.4% EPA ethyl ester, measured as a weight percent of oil, and substantially free of DHA, could readily be converted to yield an EPA concentrate in an alternate form (i.e., the EPA ethyl ester could be converted to free fatty acids, triacylglycerols, methyl esters, and combinations thereof), using means well known to those of skill in the art. Thus, for example, the 82.8% EPA ethyl ester or 95.4% EPA ethyl ester could be re-esterified to triglycerides via glycerolysis, to result in an EPA concentrate, in triglyceride form, comprising at least 70 wt % of EPA, measured as a wt % of oil, and substantially free of DHA.

### Example 4

### Enrichment of Microbial Oil Via Supercritical Fluid Chromatography

This example demonstrates that an EPA concentrate comprising up to 89.8% EPA ethyl esters, measured as a weight percent of oil, and substantially free of DHA could be obtained upon enrichment of the non-concentrated purified oil from Example 1 using a supercritical fluid chromatographic [“SFC”]method.

The non-concentrated purified oil from Example 1 was transesterified to ethyl esters using sodium ethoxide as a base catalyst, and then processed through an adsorption column to remove compounds that were insoluble in supercritical CO₂. The processed ethyl ester oil was then purified by K.D. Pharma (Bexbach, Germany) using their supercritical chromatographic technology. Various degrees of enrichment were achieved (e.g., see exemplary data for Sample #1 and Sample #2, infra). Thus, enrichment of the non-concentrated purified oil via SFC yielded an EPA concentrate with 85% and 89.8% EPA ethyl esters, measured as a weight percent of oil, and substantially free of DHA, as shown in Table 6.

TABLE 6
EPA Ethyl Ester Concentrate With SFC Enrichment Method

| Fatty | Weight Percent Of Oil | |
|---|---|---|
| acid ethyl esters | Sample #1 | Sample #2 |
| C18:2 (omega-6) | 0.4 | 0.2 |
| C20:5 EPA | 85 | 89.8 |
| C22:6 DHA | Non-detectable (<0.05%) | non-detectable (<0.05%) |
| Other components | 14.6 | 10 |

One of skill in the art will appreciate that the EPA concentrate, comprising either 85% EPA ethyl ester or 89.8% EPA ethyl ester, measured as a weight percent of oil, and substantially free of DHA, could readily be converted to yield an EPA concentrate in an alternate form (i.e., the EPA ethyl ester could be converted to free fatty acids, triacylglycerols, methyl esters, and combinations thereof), using means well known to those of skill in the art. Thus, for example, the 85% EPA ethyl ester or 89.8% EPA ethyl ester could be re-esterified to triglycerides via glycerolysis, to result in an EPA concentrate, in triglyceride form, comprising at least 70 wt % of EPA, measured as a wt % of oil, and substantially free of DHA.

### Example 5

### Preparation of a Microbial Oil Comprising 56.1% EPA of Total Fatty Acids [“TFAs”]

The present Example describes the isolation of a microbial oil obtained from microbial biomass of recombinant Yarrowia lipolytica cells, engineered for the production of EPA. This microbial oil was then enriched by fractional distillation, as described infra in Example 6.

Specifically, Y. lipolytica strain Z1978 was recombinantly engineered to enable production of about 58.7 EPA % TFAs and cultured using a 2-stage fed-batch process. Microbial oil was then isolated from the biomass via drying, extracted (via a combination of extrusion, pelletization and supercritical fluid extraction), and purified via short path distillation, yielding a non-concentrated, triglyceride-rich SPD-purified oil comprising 56.1 EPA % TFAs.

Genotype of Yarrowia lipolytica Strain Y9502

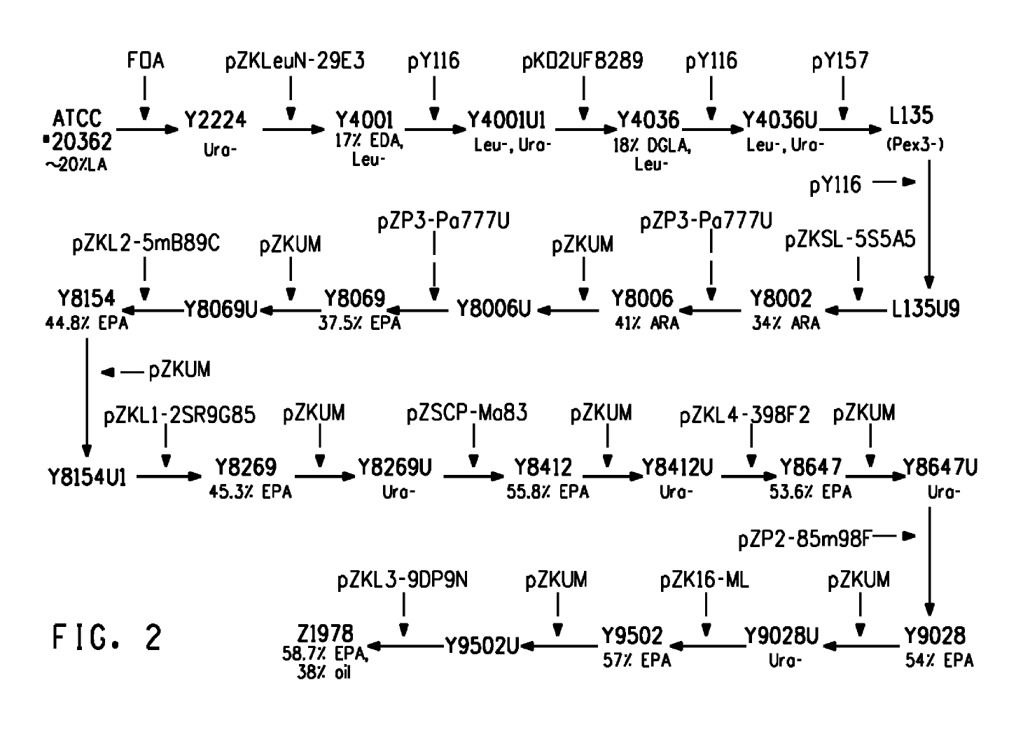

The generation of strain Y9502 is described in U.S. Pat. Appl. Pub. No. 2010-0317072-A1. Strain Y9502, derived from Yarrowia lipolytica ATCC #20362, was capable of producing about 57.0% EPA relative to the total lipids via expression of a delta-9 elongase/delta-8 desaturase pathway (FIG. 2).

The final genotype of strain Y9502 with respect to wildtype Yarrowia lipolytica ATCC #20362 was Ura+, Pex3-, unknown 1-, unknown 2-, unknown 3-, unknown 4-, unknown 5-, unknown6-, unknown 7-, unknown 8-, unknown9-, unknown 10-, YAT1::ME3S::Pex16, GPD::ME3S::Pex20, YAT1::ME3S::Lip1, FBAINm::EgD9eS::Lip2, EXP1::EgD9eS::Lip1, GPAT::EgD9e::Lip2, YAT1::EgD9eS::Lip2, FBAINm::EgD8M::Pex20, EXP1::EgD8M::Pex16, FBAIN::EgD8M::Lip1, GPD::EaD8S::Pex16 (2 copies), YAT1::E389D9eS/EgD8M::Lip1, YAT1::EgD9eS/EgD8M::Aco, FBAINm::EaD9eS/EaD8S::Lip2, GPD::FmD12::Pex20, YAT1::FmD12::Oct, EXP1::FmD12S::Aco, GPDIN::FmD12::Pex16, EXP1::EgD5M::Pex16, FBAIN::EgD5SM::Pex20, GPDIN::EgD5SM::Aco, GPM::EgD5SM::Oct, EXP1::EgD5SM::Lip1, YAT1::EaD5SM::Oct, FBAINm::PaD17::Aco, EXP1::PaD17::Pex16, YAT1::PaD17S::Lip1, YAT1::YICPT::Aco, YAT1::MCS::Lip1, FBA::MCS::Lip1, YAT1::MaLPAAT1S::Pex16.

Abbreviations not defined in Example 1 are as follows: EaD9eS/EgD8M is a DGLA synthase created by linking a codon-optimized delta-9 elongase gene (“EaD9eS”), derived from Euglena anabaena delta-9 elongase [U.S. Pat. No. 7,794,701] to the delta-8 desaturase “EgD8M” (supra) [U.S. Pat. Appl. Pub. No. 2008-0254191-A1]; and, MaLPAAT1S is a codon-optimized lysophosphatidic acid acyltransferase gene, derived from Mortierella alpina [U.S. Pat. No. 7,879,591].

For a detailed analysis of the total lipid content and composition in strain Y9502, a flask assay was conducted wherein cells were grown in 2 stages for a total of 7 days. Based on analyses, strain Y9502 produced 3.8 g/L dry cell weight [“DCW”], total lipid content of the cells was 37.1 [“TFAs % DCW”], the EPA content as a percent of the dry cell weight [“EPA % DCW”] was 21.3, and the lipid profile was as follows, wherein the concentration of each fatty acid is as a weight percent of TFAs [“% TFAs”]: 16:0 (palmitate)-2.5, 16:1 (palmitoleic acid)—0.5, 18:0 (stearic acid)—2.9, 18:1 (oleic acid)—5.0, 18:2 (LA)-12.7, ALA—0.9, EDA—3.5, DGLA—3.3, ARA—0.8, ETrA—0.7, ETA—2.4, EPA—57.0, other—7.5.

Generation of Yarrowia lipolytica Strain Z1978 from Strain Y9502

The development of strain Z1978 from strain Y9502 is described in U.S. patent application Ser. Nos. 13/218,591 (Attorney Docket Number CL4783USNA, filed Aug. 26, 2011) and No. 13/218,708 (Attorney Docket Number CL5411USNA, filed on Aug. 26, 2011), hereby incorporated herein by reference (see also FIG. 2 herein).

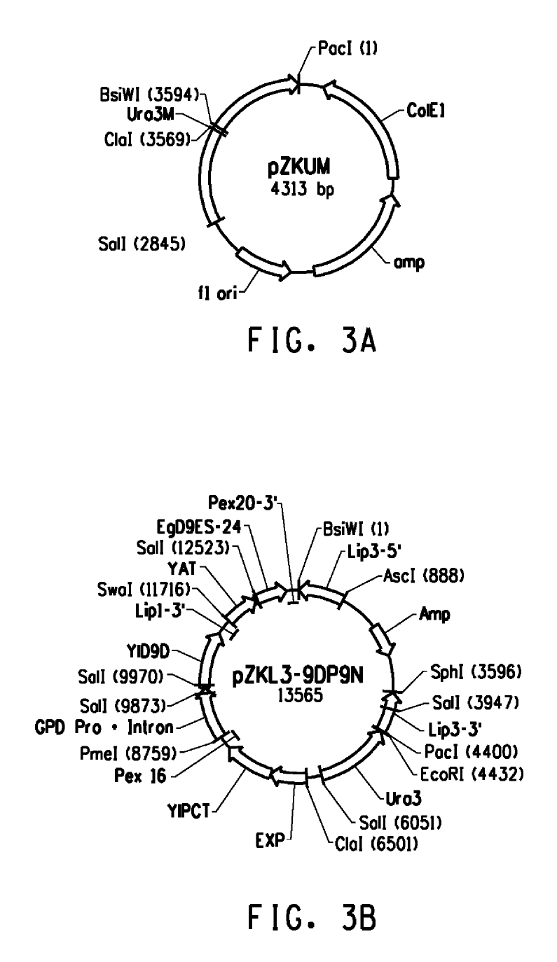

Specifically, to disrupt the Ura3 gene in strain Y9502, construct pZKUM (FIG. 3A; SEQ ID NO:1; described in Table 15 of U.S. Pat. Appl. Pub. No. 2009-0093543-A1) was used to integrate an Ura3 mutant gene into the Ura3 gene of strain Y9502. Transformation was performed according to the methodology of U.S. Pat. Appl. Pub. No. 2009-0093543-A1, hereby incorporated herein by reference. A total of 27 transformants (selected from a first group comprising 8 transformants, a second group comprising 8 transformants, and a third group comprising 11 transformants) were grown on 5-fluoroorotic acid [“FOA”] plates (FOA plates comprise per liter: 20 g glucose, 6.7 g Yeast Nitrogen base, 75 mg uracil, 75 mg uridine and appropriate amount of FOA (Zymo Research Corp., Orange, Calif.), based on FOA activity testing against a range of concentrations from 100 mg/L to 1000 mg/L (since variation occurs within each batch received from the supplier)). Further experiments determined that only the third group of transformants possessed a real Ura− phenotype.

For fatty acid [“FA”] analysis, cells were collected by centrifugation and lipids were extracted as described in Bligh, E. G. & Dyer, W. J. (Can. J. Biochem. Physiol., 37:911-917 (1959)). Fatty acid methyl esters [“FAMEs”] were prepared by transesterification of the lipid extract with sodium methoxide (Roughan, G., and Nishida I., Arch Biochem Biophys., 276(1):38-46 (1990)) and subsequently analyzed with a Hewlett-Packard 6890 GC fitted with a 30-m×0.25 mm (i.d.) HP-INNOWAX (Hewlett-Packard) column. The oven temperature was from 170° C. (25 min hold) to 185° C. at 3.5° C./min.

For direct base transesterification, Yarrowia cells (0.5 mL culture) were harvested, washed once in distilled water, and dried under vacuum in a Speed-Vac for 5-10 min. Sodium methoxide (100 μl of 1%) and a known amount of C15:0 triacylglycerol (C15:0 TAG; Cat. No. T-145, Nu-Check Prep, Elysian, Minn.) was added to the sample, and then the sample was vortexed and rocked for 30 min at 50° C. After adding 3 drops of 1 M NaCl and 400 μl hexane, the sample was vortexed and spun. The upper layer was removed and analyzed by GC (supra). FAME peaks recorded via GC analysis were identified and quantitated according to the methodology of Example 1, as was the lipid profile.

Alternately, a modification of the base-catalysed transesterification method described in Lipid Analysis, William W. Christie, 2003 was used for routine analysis of the broth samples from either fermentation or flask samples. Specifically, broth samples were rapidly thawed in room temperature water, then weighed (to 0.1 mg) into a tarred 2 mL microcentrifuge tube with a 0.22 μm Corning® Costar® Spin-X® centrifuge tube filter (Cat. No. 8161). Sample (75-800 μl) was used, depending on the previously determined DCW. Using an Eppendorf 5430 centrifuge, samples are centrifuged for 5-7 min at 14,000 rpm or as long as necessary to remove the broth. The filter was removed, liquid was drained, and −500 μl of deionized water was added to the filter to wash the sample. After centrifugation to remove the water, the filter was again removed, the liquid drained and the filter re-inserted. The tube was then re-inserted into the centrifuge, this time with the top open, for ˜3-5 min to dry. The filter was then cut approximately ½ way up the tube and inserted into a fresh 2 mL round bottom Eppendorf tube (Cat. No. 22 36 335-2).

The filter was pressed to the bottom of the tube with an appropriate tool that only touches the rim of the cut filter container and not the sample or filter material. A known amount of C15:0 TAG (supra) in toluene was added and 500 μl of freshly made 1% sodium methoxide in methanol solution. The sample pellet was firmly broken up with the appropriate tool and the tubes were closed and placed in a 50° C. heat block (VWR Cat. No. 12621-088) for 30 min. The tubes were then allowed to cool for at least 5 min. Then, 400 μl of hexane and 500 μl of a 1 M NaCl in water solution were added, the tubes were vortexed for 2×6 sec and centrifuged for 1 min. Approximately 150 μl of the top (organic) layer was placed into a GC vial with an insert and analyzed by GC.

FAME peaks recorded via GC analysis were identified by their retention times, when compared to that of known fatty acids, and quantitated by comparing the FAME peak areas with that of the internal standard (C15:0 TAG) of known amount. Thus, the approximate amount (g) of any fatty acid FAME [“μg FAME”] is calculated according to the formula: (area of the FAME peak for the specified fatty acid/area of the standard FAME peak)*(g of the standard C15:0 TAG), while the amount (g) of any fatty acid [“μg FA”] is calculated according to the formula: (area of the FAME peak for the specified fatty acid/area of the standard FAME peak)*(g of the standard C15:0 TAG)*0.9503, since 1 g of C15:0 TAG is equal to 0.9503 g fatty acids. Note that the 0.9503 conversion factor is an approximation of the value determined for most fatty acids, which range between 0.95 and 0.96.

The lipid profile, summarizing the amount of each individual fatty acid as a wt % of TFAs, was determined by dividing the individual FAME peak area by the sum of all FAME peak areas and multiplying by 100.

In this way, GC analyses showed that there were 28.5%, 28.5%, 27.4%, 28.6%, 29.2%, 30.3% and 29.6% EPA of TFAs in pZKUM-transformants #1, #3, #6, #7, #8, #10 and #11 of group 3, respectively.

These seven strains were designated as strains Y9502U12, Y9502U14, Y9502U17, Y9502U18, Y9502U19, Y9502U21 and Y9502U22, respectively (collectively, Y9502U).

Construct pZKL3-9DP9N (FIG. 3B; SEQ ID NO:2) was then generated to integrate one delta-9 desaturase gene, one choline-phosphate cytidylyl-transferase gene, and one delta-9 elongase mutant gene into the Yarrowia YALIOF32131p locus (GenBank Accession No. XM_{—}506121) of strain Y9502U. The pZKL3-9DP9N plasmid contained the following components:

TABLE 7
Description of Plasmid pZKL3-9DP9N (SEQ ID NO: 2)

| RE Sites And | |
|---|---|
| Nucleotides | |
| Within SEQ ID | Description Of |
| NO: 2 | Fragment And Chimeric Gene Components |
| AscI/BsiWI | 884 by 5′ portion of YALI0F32131p locus (GenBank |
| (887-4) | Accession No. XM_506121, labeled as |
| | “Lip3-5” in Figure) |
| PacI/SphI | 801 by 3′ portion of YALI0F32131p locus (GenBank |
| (4396-3596) | Accession No. XM_506121, labeled |
| | as “Lip3-3” in Figure) |
| SwaI/BsiWI | YAT1::EgD9eS-L35G::Pex20, comprising: |
| (11716-1) | YAT1: Yarrowia lipolytica YAT1 promoter (labeled |
| | as “YAT” in Figure; U.S. Pat. Appl. Pub. |
| | No. 2010-0068789A1); EgD9eS-L35G: Synthetic |
| | mutant of delta-9 elongase gene (SEQ ID |
| | NO: 3; U.S Pat. Appl. No. 13/218,591), derived |
| | from Euglena gracilis (“EgD9eS”; U.S. |
| | Pat. No. 7,645,604); Pex20: Pex20 terminator |
| | sequence from Yarrowia Pex20 gene (GenBank |
| | Accession No. AF054613) |
| PmeI/SwaI | GPDIN::YID9::Lip1, comprising: |
| (8759-11716) | GPDIN: Yarrowia lipolytica GPDIN promoter |
| | (U.S. Pat. No. 7,459,546); |
| | YID9: Yarrowia lipolytica delta-9 desaturase gene |
| | (GenBank Accession No. XM_501496; |
| | SEQ ID NO: 5); Lip1: Lip1 terminator sequence |
| | from Yarrowia Lip1 gene (GenBank Accession |
| | No. Z50020) |
| ClaII/PmeI | EXP::YIPCT::Pex16, comprising: |
| (6501-8759) | EXP1: Yarrowia lipolytica export protein (EXP1) |
| | promoter (labeled as “Exp” in Figure; U.S |
| | Pat. No. 7,932,077); YIPCT: Yarrowia lipolytica |
| | choline-phosphate cytidylyl-transferase [“PCT”] |
| | gene (Gen Bank Accession No. XM_502978; |
| | SEQ ID NO: 7); |
| | Pex16: Pex16 terminator sequence from Yarrowia |
| | Pex16 gene (GenBank Accession No. U75433) |
| SalI/EcoRI | Yarrowia Ura3 gene (Gen Bank Accession |
| (6501-4432) | No. AJ306421) |

The pZKL3-9DP9N plasmid was digested with AscI/SphI, and then used for transformation of strain Y9502U17. The transformant cells were plated onto Minimal Media [“MM”] plates and maintained at 30° C. for 3 to 4 days (Minimal Media comprises per liter: 20 g glucose, 1.7 g yeast nitrogen base without amino acids, 1.0 g proline, and pH 6.1 (do not need to adjust)). Single colonies were re-streaked onto MM plates, and then inoculated into liquid MM at 30° C. and shaken at 250 rpm/min for 2 days. The cells were collected by centrifugation, resuspended in High Glucose Media [“HGM”] and then shaken at 250 rpm/min for 5 days (High Glucose Media comprises per liter: 80 glucose, 2.58 g KH₂ PO₄ and 5.36 g K₂HPO₄, pH 7.5 (do not need to adjust)). The cells were subjected to fatty acid analysis, supra.

GC analyses showed that most of the selected 96 strains of Y9502U17 with pZKL3-9DP9N produced 50-56% EPA of TFAs. Five strains (i.e., #31, #32, #35, #70 and #80) that produced about 59.0%, 56.6%, 58.9%, 56.5%, and 57.6% EPA of TFAs were designated as Z1977, Z1978, Z1979, Z1980 and Z1981 respectively.

The final genotype of these pZKL3-9DP9N transformant strains with respect to wildtype Yarrowia lipolytica ATCC #20362 was Ura+, Pex3-, unknown 1-, unknown 2-, unknown 3-, unknown 4-, unknown 5-, unknown6-, unknown 7-, unknown 8-, unknown9-, unknown 10-, unknown 11-, YAT1::ME3S::Pex16, GPD::ME3S::Pex20, YAT1::ME3S::Lip1, FBAINm::EgD9eS::Lip2, EXP1::EgD9eS::Lip1, GPAT::EgD9e::Lip2, YAT1::EgD9eS::Lip2, YAT::EgD9eS-L35G::Pex20, FBAINm::EgD8M::Pex20, EXP1::EgD8M::Pex16, FBAIN::EgD8M::Lip1, GPD::EaD8S::Pex16 (2 copies), YAT1::E389D9eS/EgD8M::Lip1, YAT1::EgD9eS/EgD8M::Aco, FBAINm::EaD9eS/EaD8S::Lip2, GPDIN::YID9::Lip1, GPD::FmD12::Pex20, YAT1::FmD12::Oct, EXP1::FmD12S::Aco, GPDIN::FmD12::Pex16, EXP1::EgD5M::Pex16, FBAIN::EgD5SM::Pex20, GPDIN::EgD5SM::Aco, GPM::EgD5SM::Oct, EXP1::EgD5SM::Lip1, YAT1::EaD5SM::Oct, FBAINm::PaD17::Aco, EXP1::PaD17::Pex16, YAT1::PaD17S::Lip1, YAT1::YICPT::Aco, YAT1::MCS::Lip1, FBA::MCS::Lip1, YAT1::MaLPAAT1S::Pex16, EXP1::YIPCT::Pex16.

Knockout of the YALIOF32131p locus (GenBank Accession No. XM_{—}50612) in strains Z1977, Z1978, Z1979, Z1980 and Z1981 was not confirmed in any of these EPA strains produced by transformation with pZKL3-9DP9N.

Cells from YPD plates of strains Z1977, Z1978, Z1979, Z1980 and Z1981 were grown and analyzed for total lipid content and composition, according to the methodology below.

For a detailed analysis of the total lipid content and composition in a particular strain of Y. lipolytica, flask assays were conducted as follows. Specifically, one loop of freshly streaked cells was inoculated into 3 mL Fermentation Medium [“FM”] medium and grown overnight at 250 rpm and 30° C. (Fermentation Medium comprises per liter: 6.70 g/L yeast nitrogen base, 6.00 g KH₂ PO₄, 2.00 g K₂HPO₄, 1.50 g MgSO₄*7H₂O, 20 g glucose and 5.00 g yeast extract (BBL)). The OD₆₀₀ₙₘ was measured and an aliquot of the cells were added to a final OD₆₀₀ₙₘ of 0.3 in 25 mL FM medium in a 125 mL flask. After 2 days in a shaker incubator at 250 rpm and at 30° C., 6 mL of the culture was harvested by centrifugation and resuspended in 25 mL HGM in a 125 mL flask. After 5 days in a shaker incubator at 250 rpm and at 30° C., a 1 mL aliquot was used for fatty acid analysis (supra) and 10 mL dried for dry cell weight [“DCW”] determination.

For DCW determination, 10 mL culture was harvested by centrifugation for 5 min at 4000 rpm in a Beckman GH-3.8 rotor in a Beckman GS-6R centrifuge. The pellet was resuspended in 25 mL of water and re-harvested as above. The washed pellet was re-suspended in 20 mL of water and transferred to a pre-weighed aluminum pan. The cell suspension was dried overnight in a vacuum oven at 80° C. The weight of the cells was determined.

Total lipid content of cells [“TFAs % DCW”] is calculated and considered in conjunction with data tabulating the concentration of each fatty acid as a weight percent of TFAs [“% TFAs”] and the EPA content as a percent of the dry cell weight [“EPA % DCW”].

Thus, Table 8 below summarizes total lipid content and composition of strains Z1977, Z1978, Z1979, Z1980 and Z1981, as determined by flask assays. Specifically, the Table summarizes the total dry cell weight of the cells [“DCW”], the total lipid content of cells [“TFAs % DCW”], the concentration of each fatty acid as a weight percent of TFAs [“% TFAs”] and the EPA content as a percent of the dry cell weight [“EPA % DCW”].

TABLE 8
Total Lipid Content And Composition In Yarrowia Strains Z1977, Z1978, Z1979, Z1980 and Z1981 By Flask Assay

| | DCW | TFAs % | % TFAs | | | | | | | | | | | | | EPA % |
|---|---|---|---|---|---|---|---|---|---|---|---|---|---|---|---|---|
| Strain | (g/L) | DCW | 16:0 | 16:1 | 18:0 | 18:1 | 18:2 | ALA | EDA | DGLA | ARA | EtrA | ETA | EPA | other | DCW |
| Z1977 | 3.8 | 34.3 | 2.0 | 0.5 | 1.9 | 4.6 | 11.2 | 0.7 | 3.1 | 3.3 | 0.9 | 0.7 | 2.2 | 59.1 | 9.9 | 20.3 |
| Z1978 | 3.9 | 38.3 | 2.4 | 0.4 | 2.4 | 4.8 | 11.1 | 0.7 | 3.2 | 3.3 | 0.8 | 0.6 | 2.1 | 58.7 | 9.5 | 22.5 |
| Z1979 | 3.7 | 33.7 | 2.3 | 0.4 | 2.4 | 4.1 | 10.5 | 0.6 | 3.2 | 3.6 | 0.9 | 0.6 | 2.2 | 59.4 | 9.8 | 20.0 |
| Z1980 | 3.6 | 32.7 | 2.1 | 0.4 | 2.2 | 4.0 | 10.8 | 0.6 | 3.1 | 3.5 | 0.9 | 0.7 | 2.2 | 59.5 | 10.0 | 19.5 |
| Z1981 | 3.5 | 34.3 | 2.2 | 0.4 | 2.1 | 4.2 | 10.6 | 0.6 | 3.3 | 3.4 | 1.0 | 0.8 | 2.2 | 58.5 | 10.7 | 20.1 |

Strain Z1978 was subsequently subjected to partial genome sequencing (U.S. patent application Ser. No. 13/218,591). This work determined that four (not six) delta-5 desaturase genes were integrated into the Yarrowia genome (i.e., EXP1::EgD5M::Pex16, FBAIN::EgD5SM::Pex20, EXP1::EgD5SM::Lip1, and YAT1::EaD5SM::Oct).

Fermentation and Disruption Via Extrusion and Pelletization of Dried, Untreated Y. lipolytica Strain Z1978 Biomass

A Y. lipolytica strain Z1978 culture was fermented and the microbial biomass was harvested and dried, as described in Example 1. The dried and untreated biomass was then fed to a twin screw extruder. Specifically, a mixture of the biomass and 15% of diatomaceous earth (Celatom MN-4 or Celite 209, EP Minerals, LLC, Reno, Nev.) were premixed and then fed to a ZSK-40 mm MC twin screw extruder (Coperion Werner & Pfleiderer, Stuttgart, Germany) at a rate of 45.5 kg/hr. A water/sucrose solution made of 26.5% sucrose was injected after the disruption zone of the extruder at a flow rate of 147 mL/min. The extruder was operated at 280 rpm with a % torque range of 20-23. The resulting disrupted yeast powder was cooled to 35° C. in a final water cooled barrel. The moist extruded powder was then fed into a LCI Dome Granulator Model No. TDG-80 (LCI Corporation, Charlotte, N.C.) assembled with a multi-bore dome die 1 mm diameter by 1 mm thick screen and set to 82 RPM. Extrudate was formed at 455-600 kg/hr (as dried rate). The sample was dried in a vibratory fluid bed dryer (FBP-75, Carman Industries, Inc., Jeffersonville, Ind.) with a drying zone of 0.50 m² with 1150 standard cubic feet per minute [“scfm”] of air flow maintained at 100° C. and a cooling zone of 0.24 m² operating with an air flow estimated at 500-600 scfm at 18° C. Dried pellets, approximately 1 mm diameter×6 to 10 mm in length, exited the dryer in the 25-30° C. range, having a final moisture content of 5-6% measured on an O'Haus moisture analyzer (Parsippany, N.J.).

### Oil Extraction of the Extruded Yeast Biomass

The extruded yeast pellets were extracted using supercritical fluid phase carbon dioxide (CO₂) as the extraction solvent to produce non-concentrated extracted oil. Specifically, the yeast pellets were charged to a 320 L stainless steel extraction vessel and packed between plugs of polyester foam filtration matting (Aero-Flo Industries, Kingsbury, Ind.). The vessel was sealed, and then CO₂ was metered by a commercial compressor (Pressure Products Industries, Warminster, Pa.) through a heat exchanger (pre-heater) and fed into the vertical extraction vessel to extract the non-concentrated extracted oil from the pellets of disrupted yeast. The extraction temperature was controlled by the pre-heater, and the extraction pressure was maintained with an automated control valve (Kammer) located between the extraction vessel and a separator vessel. The CO₂ and oil extract was expanded to a lower pressure through this control valve. Oil extract was collected from the expanded solution as a precipitate in the separator. The temperature of the expanded CO₂ phase in the separator was controlled by use of an additional heat exchanger located upstream of the separator. This lower pressure CO₂ stream exited the top of the separator vessel and was recycled back to the compressor through a filter, a condenser, and a mass flow meter. The oil extract was periodically drained from the separator and collected as product.

The extraction vessel was initially charged with approximately 150 kg of the extruded yeast pellets. The non-concentrated extracted oil was then extracted from the pellets with supercritical fluid CO₂ at 5000 psig (345 bar), 55° C., and a solvent-to-feed ratio ranging from 40 to 50 kg CO₂ per kg of starting yeast pellets. Roughly 37.5 kg of non-concentrated extracted oil was collected from the separator vessel, to which was added about 1000 ppm each of two antioxidants, i.e. Covi-ox T70 (Cognis, Mississauga, Canada) and Dadex RM (Nealanders, Mississauga, Canada).

### Distillation Under SPD Conditions

The non-concentrated extracted oil was degassed and then passed through a 6″ molecular still (POPE Scientific, Saukville, Wis.) using a feed rate of 12 kg/hr to remove residual water. The surface temperatures of the evaporator and condenser were set at 140° C. and 15° C., respectively. The vacuum was maintained at 15 torr.

The dewatered extracted oil was passed through the molecular still at a feed rate of 12 kg/hr for a second time to remove undesired lower-molecular weight compounds, such as ergosterol and free fatty acids in the distillate. The vacuum was lowered to 1 mtorr, and the surface temperatures of the evaporator were maintained between 240° C. and 270° C. A triacylglycerol-containing fraction (i.e., the SPD-purified oil) was obtained, having reduced sterols relative to the sterol content in the non-concentrated extracted oil. The non-concentrated SPD-purified oil was cooled to below 40° C. before packaging.

Characterization of SPD-Purified Oil from Yarrowia lipolytica Strain Z1978

The fatty acid composition of the non-concentrated SPD-purified oil from strain Z1978 was analyzed, following transesterification, according to the methodology of Example 1. The SPD-purified oil contained 56.1 EPA % TFAs and DHA was non-detectable (i.e. <0.05%), as shown below in Table 9.

TABLE 9
Fatty Acid Composition Of Non-Concentrated Z1978 SPD-
Purified Oil

| Fatty acid | Weight Percent Of Total Fatty Acids |
|---|---|
| C18:2 (omega-6) | 14.2 |
| C20:5 EPA | 56.1 |
| C22:6 DHA | non-detectable (<0.05%) |
| Other components | 29.7 |

### Example 6

### Enrichment of Microbial Oil Via Fractional Distillation

This example demonstrates that an EPA concentrate comprising up to 74% EPA ethyl ester, measured as a weight percent of oil, and substantially free of DHA could be obtained upon enrichment of the non-concentrated SPD-purified oil from Example 5 using a fractional distillation method.

Twenty-five (25) kg of the non-concentrated microbial oil from Example 5 was added to a 50 L glass flask. 7.9 kg of absolute ethanol and 580 g of sodium ethoxide (21% in ethanol) were then added to the flask. The mixture was heated to reflux at −85° C. for a minimum of 30 min. The reaction was monitored by a thin layer chromatography method, where a diluted sample of the oil was spotted onto a silica plate and separated using an acetic acid/hexane/ethyl ether solvent mixture. Spots consisting of unreacted TAGs were detected by iodine stain. Absent or barely detectable spots were considered to represent completion of the reaction. After the reaction end point was reached, the mixture was cooled to below 50° C. and allowed to phase separate. The glycerol-containing bottom layer was separated and discarded. The upper organic layer was washed with 2.5 L of 5% citric acid, and the recovered organic layer was then washed with 5 L of 15% aqueous sodium sulfate. The aqueous phase was again discarded, and the ethyl ester phase was distilled with ethanol in a rotavap at −60° C. to remove residual water. Approximately 25 kg of oil in ethyl ester form was recovered.

The ethyl esters were then fed to a 4″ hybrid wiped-film and fractionation system (POPE Scientific, Saukville, Wis.) at a feed rate of 5 kg/hr to enrich EPA ethyl esters. The evaporator temperature was set at approximately 275° C. under a vacuum of 0.47 torr. The head temperature of the packed column was about 146° C. The lower-molecular-weight ethyl esters, mainly C18s, were removed as a light fraction from the overhead. The extracted EPA ethyl esters were recovered as a heavy fraction and underwent a second distillation, mainly for removing color and polymerized. The second distillation was performed in a 6″ molecular still (POPE Scientific, Saukville, Wis.) at a feed rate of 20 kg/hr. The evaporator was operated at about 205° C. with an internal condenser temperature setting of about 10° C. and a vacuum of 0.01 torr. Approximately 7-10 wt % of the ethyl esters was removed, yielding a clear and light color EPA concentrate. The final EPA concentrate contained 74% EPA ethyl esters, measured as a weight percent of oil, and substantially free of DHA.

One of skill in the art will appreciate that the EPA concentrate, comprising 74% EPA ethyl ester, measured as a weight percent of oil, and substantially free of DHA, could readily be converted to yield an EPA concentrate in an alternate form (i.e., the EPA ethyl ester could be converted to free fatty acids, triacylglycerols, methyl esters, and combinations thereof), using means well known to those of skill in the art. Thus, for example, the 74% EPA ethyl ester could be re-esterified to triglycerides via glycerolysis, to result in an EPA concentrate, in triglyceride form, comprising at least 70 wt % of EPA, measured as a wt % of oil, and substantially free of DHA.

### Example 7

### EPA Concentrates are Substantially Free of Environmental Pollutants

This example demonstrates that both an EPA concentrate comprising at least 70 wt % of EPA, measured as a wt % of oil, and substantially free of DHA, and the microbial oil comprising 30-70 wt % of EPA, measured as a wt % of TFAs, and substantially free of DHA, are substantially free of environmental pollutants.

A comparable sample of non-concentrated purified oil from Yarrowia lipolytica strain Y8672 was prepared, as described in Example 1. The concentration, measured as mg/g World Health Organization International Toxicity Equivalent [“WHO TEQ”], of polychlorinated biphenyls [“PCBs”](CAS No. 1336-36-3), polychlorinated dibenzodioxins [“PCDDs”] and polychlorinated dibenzofurans [“PCDFs”] in the non-concentrated extracted oil was determined according to EPA method 1668 Rev A. Extremely low or non-detectable levels of the environmental pollutants were detected.

Based on the results above, it is assumed herein that the concentration of PCBs, PCDDs, and PCDFs in the non-concentrated extracted oil of Example 1 and the non-concentrated SPD-purified oil of Example 5 will also contain extremely low or non-detectable levels of environmental pollutants. Similarly, it is hypothesized herein that the EPA ethyl ester concentrates in Examples 2, 3, 4 and 6, enriched via urea adduct formation, liquid chromatography, SFC and fractional distillation, respectively, should also contain extremely low or non-detectable levels of environmental pollutants since they were produced from non-concentrated oils that are themselves substantially free of environmental pollutants.

More specifically, Table 10 describes the expected TEQ levels of PCBs, PCDDs, and PCDFs within the EPA concentrates in Examples 2, 3, 4 and 6. For comparison, the concentrations of the same compounds in a pollutant-stripped marine oil described in U.S. Pat. No. 7,732,488 are also included. It is noted that U.S. Pat. No. 7,732,488 provides special processing methods to reduce these environmental pollutants to acceptable levels.

TABLE 10
Expected Environmental Pollutant Concentration (pg/g WHO TEQ) In
EPA Concentrates

| | EPA ethyl ester | FIG. 2 from U.S. Pat. |
|---|---|---|
| | concentrates | No. 7,732,488 |
| Polychlorinated Biphenyls | <0.1 | 0.17 |
| (PCBs) | | |
| Polychlorinated Dibenzodioxins | <0.1 | 0.26 |
| (PCDDs, dioxins) | | |
| Polychlorinated Dibenzofurans | non-detectable | 0.2 |
| (PCDFs, furans) | (<0.03) | |

As shown above, the EPA ethyl ester concentrates in Examples 2, 3, 4 and 6 will have lower levels of PCBs, PCDDs and PCDFs than the pollutant-stripped marine oil in U.S. Pat. No. 7,732,488. In fact, the pollutant level of PCDFs is expected to be below the detection limit of the analytical method used.

### Example 8

### Enrichment of Microbial Oil Via Fractional Distillation and Liquid Chromatography

This example demonstrates that an EPA concentrate comprising up to 97.4% EPA ethyl ester, measured as a weight percent of oil, and substantially free of DHA, NDPA and HPA could be obtained upon enrichment of a non-concentrated purified oil using a combination of fractional distillation and liquid chromatography methods.

Non-concentrated purified oil was obtained from Yarrowia lipolytica strain Y9502 (supra, Example 5; see also U.S. Pat. Appl. Pub. No. 2010-0317072-A1). Specifically, the strain was cultured, harvested, disrupted via extrusion and pelletization, and extracted using supercritical fluid phase CO₂ as described in Example 5. The non-concentrated extracted oil was then purified under SPD conditions (Example 5).

Characterization of SPD-Purified Oil from Yarrowia lipolytica Strain Y9502

The fatty acid composition of the non-concentrated SPD-purified oil from strain Y9502 was analyzed according to the methodology of Example 1. The SPD-purified oil contained 54.7 EPA % TFAs and DHA, NDPA and HPA were non-detectable (i.e., <0.05%), as shown below in Table 11.

TABLE 11
Fatty Acid Composition Of Non-Concentrated Y9502 SPD-Purified Oil

| Fatty acid | Weight Percent Of Total Fatty Acids |
|---|---|
| C18:2 (omega-6) | 15 |
| C19:5 (omega-2) | non-detectable (<0.05%) |
| C20:5 EPA | 54.7 |
| C21:5 HPA | Non-detectable (<0.05%) |
| C22:6 DHA | non-detectable (<0.05%) |
| Other components | 30.3 |

Enrichment of SPD-Purified Oil from Yarrowia lipolytica Strain Y9502

The SPD-purified oil was transesterified to ethyl esters using a similar method as described in Example 3 and further subjected to fractional distillation as described in Example 5. The fractionally distilled EPA concentrate contained 71.9% EPA ethyl esters, measured as a weight percent of oil, and was substantially free of DHA, NDPA and HPA (see the column titled “Fractionally Distilled” below in Table 12).

The fractionally distilled ethyl esters were then enriched by Equateq (Isle of Lewis, Scotland) using their liquid chromatographic purification technology. The enrichment of the fractionally distilled EPA concentrate via liquid chromotography yielded a final EPA concentrate with up to 97.4% EPA ethyl ester, measured as a weight percent of oil, and substantially free of DHA, NDPA and HPA (see the column titled “Liquid Chromotography Enriched” below in Table 12).

TABLE 12
EPA Ethyl Ester Concentrate With A Liquid Chromotography
Enrichment Method

| | Weight Percent Of Oil | |
|---|---|---|
| Fatty acid | | Liquid Chromotography |
| ethyl esters | Fractionally Distilled | Enriched |
| C18:2 (omega-6) | 0.8 | 0.05 |
| C19:5 NDPA | Non-detectable (<0.05%) | Non-detectable (<0.05%) |
| (omega-2) | | |
| C20:5 EPA | 71.9 | 97.4 |
| C21:5 HPA | Non-detectable (<0.05%) | Non-detectable (<0.05%) |
| C22:6 DHA | Non-detectable (<0.05%) | Non-detectable (<0.05%) |
| Other components | 27.3 | 2.1 |

One of skill in the art will appreciate that the EPA concentrate, comprising 97.4% EPA ethyl ester, measured as a weight percent of oil, and substantially free of DHA, NPDA and HPA, could readily be converted to yield an EPA concentrate in an alternate form (i.e., the EPA ethyl ester could be converted to free fatty acids, triacylglycerols, methyl esters, and combinations thereof), using means well known to those of skill in the art. Thus, for example, the 97.4% EPA ethyl ester could be re-esterified to triglycerides via glycerolysis, to result in an EPA concentrate, in triglyceride form, comprising at least 70 wt % of EPA, measured as a wt % of oil, and substantially free of DHA, NPDA and HPA.

Additionally, it is noted that EPA concentrates prepared according to the methods of the invention herein from any microbial biomass of recombinant Yarrowia cells, engineered for the production of EPA, are expected to be substantially free of DHA, NDPA and HPA. The results obtained above based on microbial oil obtained from Y. lipolytica strain Y9502, wherein the final EPA concentrate is substantially free of DHA, NDPA and HPA, would be expected from EPA concentrates prepared from microbial oils obtained from Example 1 and Example 5. Since DHA, NDPA and HPA impurities are not present in the initial microbial oil comprising 30 to 70 wt % of EPA, measured as a wt % of TFAs, obtained from a Yarrowia that accumulates in excess of 25% of its dry cell weight as oil, the fatty acid impurities will also not be present in an EPA concentrate produced therefrom.

What is claimed is:

1. An eicosapentaenoic acid concentrate comprising at least 70 weight percent of eicosapentaenoic acid, measured as a weight percent of oil, and substantially free of docosahexaenoic acid, said concentrate obtained from a microbial oil comprising 30 to 70 weight percent of eicosapentaenoic acid, measured as a weight percent of total fatty acids, and substantially free of docosahexaenoic acid;
wherein said microbial oil is obtained from a microorganism that accumulates in excess of 25% of its dry cell weight as oil.

2. The eicosapentaenoic acid concentrate of claim 1 wherein the at least 70 weight percent of eicosapentaenoic acid, measured as a weight percent of oil, is in a form selected from the group consisting of:
a) an acid, a triglyceride, an ester or combinations thereof; and,
b) an ethyl ester.

3. The eicosapentaenoic acid concentrate of claim 1 wherein the microbial oil:
a) comprises from about 1 to about 25 weight percent linoleic acid, measured as a weight percent of total fatty acids; and,
b) has a ratio of at least 1.2 of eicosapentaenoic acid, measured as a weight percent of total fatty acids, to linoleic acid, measured as a weight percent of total fatty acids.

4. The eicosapentaenoic acid concentrate of claim 1 wherein the microbial oil is obtained from microbial biomass of recombinant Yarrowia cells, engineered for the production of eicosapentaenoic acid.

5. A pharmaceutical product comprising the eicosapentaenoic acid concentrate of claim 1 or a derivative thereof.

6. A method for making an eicosapentaenoic acid concentrate comprising at least 70 weight percent of eicosapentaenoic acid, measured as a weight percent of oil, and substantially free of docosahexaenoic acid, said method comprising:
a) transesterifying a microbial oil comprising 30 to 70 weight percent of eicosapentaenoic acid, measured as a weight percent of total fatty acids, and substantially free of docosahexaenoic acid, wherein said microbial oil is obtained from a microorganism that accumulates in excess of 25% of its dry cell weight as oil; and,
b) enriching the transesterified oil of step (a) to obtain an eicosapentaenoic acid concentrate comprising at least 70 weight percent of eicosapentaenoic acid, measured as a weight percent of oil, and substantially free of docosahexaenoic acid.

7. The method of claim 6 wherein the eicosapentaenoic acid concentrate comprising at least 70 weight percent of eicosapentaenoic acid, measured as a weight percent of oil, is in a form selected from the group consisting of:
a) an acid, a triglyceride, an ester or combinations thereof; and,
b) an ethyl ester.

8. The method of claim 6 wherein the microbial oil has a ratio of at least 1.2 of eicosapentaenoic acid, measured as a weight percent of total fatty acids, to linoleic acid, measured as a weight percent of total fatty acids.

9. The method of claim 6 wherein the microbial oil is obtained from microbial biomass of recombinant Yarrowia cells, engineered for the production of eicosapentaenoic acid.

10. The method of claim 6, wherein the transesterified oil of step (a) is enriched by a process selected from the group consisting of: urea adduct formation, liquid chromatography, supercritical fluid chromatography, fractional distillation, simulated moving bed chromatography, actual moving bed chromatography and combinations thereof.

11. The method of claim 10, wherein the transesterified oil of step (a) is enriched by combination of at least two processes, said first process comprising fractional distillation.

12. The eicosapentaenoic acid concentrate of claim 1, substantially free of environmental pollutants.

13. Use of a microbial oil obtained from a microorganism that accumulates in excess of about 25% of its dry cell weight as oil, said microbial oil having 30 to 70 weight percent of eicosapentaenoic acid, measured as a weight percent of total fatty acids, and substantially free of docosahexaenoic acid, to make an eicosapentaenoic acid concentrate comprising at least 70 weight percent of eicosapentaenoic acid, measured as a weight percent of oil, and substantially free of docosahexaenoic acid.

14. The microbial oil of any one of claims 1-4, wherein the microbial oil is non-concentrated.

15. The microbial oil of any one of claims 1-4, wherein the microbial oil is substantially free of a fatty acid selected from the group consisting of nonadecapentaenoic acid and heneicosapentaenoic acid.

16. The eicosapentaenoic acid concentrate of claim 15, wherein said eicosapentaenoic acid concentrate is substantially free of a fatty acid selected from the group consisting of nonadecapentaenoic acid and heneicosapentaenoic acid.
